(19) United States
(12) Patent Application Publication
(10) Pub. No.: US 2023/0358762 A1
(43) Pub. Date: Nov. 9, 2023

(54) DIAGNOSTIC METHODS FOR KAWASAKI DISEASE

(71) Applicant: SEATTLE CHILDREN'S HOSPITAL D/B/A SEATTLE CHILDREN'S RESEARCH INSTITUTE, Seattle, WA (US)

(72) Inventors: Rhonda Fay RHYNE, Kirkland, WA (US); Craig Agamemnon MAGARET, Seattle, WA (US); Grady BARNES, Grayslake, IL (US); Celine PETERS, San Diego, CA (US); Michael PORTMAN, Seattle, WA (US)

(21) Appl. No.: 18/044,752

(22) PCT Filed: Sep. 15, 2021
(86) PCT No.: PCT/US2021/050526

§ 371 Date: Mar. 9, 2023

Related U.S. Application Data
(60) Provisional application No. 63/079,410, filed on Sep. 16, 2020.
(60) Provisional application No. 63/181,867, filed on Apr. 29, 2021.

(51) Int. Cl. G01N 33/68; G16H 50/20

(52) U.S. Cl. CPC G01N 33/6893; G16H 50/20; G01N 2800/60; G01N 2800/328

(57) ABSTRACT

Compositions and methods are provided for diagnosis, prognosis, and/or monitoring of Kawasaki disease or MIS-C (e.g., associated with SARS-CoV-2 infection) in a subject. In some embodiments, the method includes measuring, comparing and weighting the level of particular proteins to other proteins. In other embodiments, the method includes comparison with clinical variable information.

### CROSS REFERENCE TO RELATED APPLICATIONS

This application claims priority to U.S. Provisional Application No. 63/079,410, filed on Sep. 16, 2020, and U.S. Provisional Application No. 63/181,867, filed on Apr. 29, 2021, which are hereby incorporated by reference in their entireties and for all purposes.

### FIELD

The present disclosure relates protein marker panels, assays, and kits and methods for determining the diagnosis, prognosis and/or monitoring of Kawasaki disease in a patient.

### BACKGROUND

Kawasaki disease (KD) is a medium vessel vasculitis with predilection for coronary arteries. Due to lack of a reliable confirmatory laboratory test, the diagnosis of KD is based on a constellation of clinical findings that appear in a typical temporal sequence. These diagnostic criteria have been modified from time to time. However, several children may have incomplete or atypical forms of KD and the diagnosis can often be difficult, especially in infants and young children (see, for example, Ref 1).

KD is relatively uncommon, mostly affecting children under the age of 5 years but can occur in older children. It is not known what causes KD and there is currently no diagnostic test, leaving doctors to diagnose the disease based on clinical criteria (such as presence of fever, rash, swollen lymph nodes and red eyes). The most serious complication of KD is damage to the coronary arteries, potentially requiring long-term management. Rarely, children can present critically unwell with shock (low blood pressure) due to impaired heart muscle function—known as Kawasaki shock syndrome (K55), with overlapping features of toxic shock syndrome (TSS).

A need therefore exists for methods for diagnosis, prognosis, and/or monitoring of Kawasaki disease, and associated outcomes, including permanent coronary artery and heart damage in children.

### SUMMARY

In an aspect, provided herein are methods of determining Kawasaki disease or Multi-System Inflammatory Syndrome in Children (MIS-C) in a subject including (i) providing a biological sample from a subject suspected of having Kawasaki disease or MIS-C; (ii) applying the biological sample to an analytical device including (a) detecting a concentration of at least two protein markers in the biological sample; (b) calculating the concentration of the at least two protein markers against a synthetic quantification standard to produce a protein marker concentration; (c) log transforming the concentration of the at least two protein markers to conform to a normal distribution; and (d) normalizing the log-transformed concentrations of the at least two protein markers to an established range and scale, where the at least two protein markers are selected from those set forth in Table 1; (iii) optionally, determining the status of at least one clinical variable for the subject, where the clinical variable is selected from those set forth in Table 2; (iv) calculating a diagnostic score using an algorithm that applies different weightings to a transformed, normalized concentration of protein markers determined in step (ii) and, optionally, the status of the clinical variable(s) determined in step (iii); (v) classifying the diagnostic score as a positive, intermediate, or negative score; and (vi) determining Kawasaki disease or MIS-C and appropriate therapeutic intervention in the subject as indicated by the diagnostic score.

In an aspect, provided herein is a method of administering a therapeutic intervention to a subject suspected of having Kawasaki disease or MIS-C including: (i) determining the subject's protein marker profile for a panel of protein markers including at least two protein markers selected from those set forth in Table 1; (ii) optionally, determining the status of at least one clinical variable for the subject, where the clinical variable is selected from those set forth in Table 2; (iii) assigning a score to the subject based on the measured protein marker profile, and optionally the clinical value status, where the score is classified as a positive, intermediate, or negative score, said score algorithmically derived from mathematically transformed and normalized concentrations of protein markers in the subject's sample, and optionally, the status of at least one clinical variable; and (iv) administering to the subject a therapeutic intervention based on the positive, intermediate, or negative score.

In an aspect, provided herein, is a method of detecting two or more protein markers in a subject that is suspected of having Kawasaki disease or MIS-C, the method including: (i) selecting a subject that is suspected of having Kawasaki disease or MIS-C; (ii) providing a biological sample from the subject; (iii) applying the biological sample to an analytical device; (iv) detecting a concentration of at least two protein markers from Table 1; (v) calculating a diagnostic score using an algorithm that applies different weightings to the concentration of protein markers as determined by the analytical device, and, optionally, the status of the clinical variable(s); (vi) classifying the diagnostic score as a positive, intermediate, or negative score; and (vii) determining Kawasaki disease or MIS-C in a subject as indicated by the diagnostic score.

In an aspect, provided herein, is a panel for the diagnosis of Kawasaki disease or MIS-C, including target-binding agents that bind at least two protein markers selected from those listed in Table 1, a synthetic standard, and optionally, at least one clinical variable selected from those set forth in Table 2.

In an aspect, provided herein, is a panel for the diagnosis of Kawasaki disease or MIS-C, including target-binding agents for alpha-1-antitrypsin, C Reactive Protein, interleukin-1 beta, matrix metalloproteinase-9, N-terminal prohormone of brain natriuretic peptide, periostin, T3 uptake, T4 uptake, T3, T4, TSH, BNP, and thyroxine-binding globulin.

In an aspect, provided herein, is a diagnostic kit including: a panel for the diagnosis of Kawasaki disease or MIS-C, including target-binding agents that bind at least two protein markers selected from those listed in Table 1, a synthetic standard, and optionally, at least one clinical variable selected from those set forth in Table 2. In an aspect, provided herein, is a diagnostic kit including: a panel for the diagnosis of Kawasaki disease or MIS-C, including target-binding agents that bind at least two protein markers selected from those listed in Table 1, and a synthetic standard. In embodiments, the kit includes a list of clinical variables selected from those set forth in Table 2, suspicion of having MIS-C and/or SARS-CoV-2, and/or diagnosis of infection with SARS-CoV-2. In embodiments, the kit includes instructions for use of the kit. In embodiments, the instructions include instructions for assessing or including a clinical variable selected from those set forth in Table 2, suspicion of having MIS-C and/or SARS-CoV-2, and/or diagnosis of infection with SARS-CoV-2.

In an aspect, provided herein, is the use of a panel for evaluation of a subject's positive, intermediate, or negative response to a therapeutic and/or intervention for Kawasaki disease or MIS-C.

In an aspect, provided herein, is a method for treating a patient with a fever or suspected of having Kawasaki disease or MIS-C with intervention and additional testing including (i) determining whether the patient suffers from Kawasaki disease or MIS-C by: obtaining or having obtained a biological sample from the patient; performing or having performed a biomarker assay on the biological sample wherein the biomarker is selected from alpha-1 antitrypsin, C Reactive Protein, interleukin-1 beta, matrix metalloproteinase-9, N-terminal prohormone of brain natriuretic peptide, periostin and thyroxine-binding globulin; and calculating a diagnostic score based on a weighted level of biomarker; and (ii) if the patient has a positive diagnostic score, then performing additional testing or one or more interventions selected from administration of pharmacological agents or antibodies, echocardiography, avoidance of high dose acetylsalicylic acid (ASA) in patients with concomitant active infection with varicella or influenza, or enrolling in a clinical trial; if the patient has an intermediate score, then the need for further testing and on-going monitoring; if the patient has a negative score, then the need for one or more interventions selected from further testing, differential diagnosis of other diseases, including measles, other viral infections (e.g., adenovirus, enterovirus), and juvenile idiopathic arthritis or other conditions including drug hypersensitivity reactions, including Stevens Johnson syndrome.

In an aspect, provided herein are panels for the diagnosis, prognosis, and/or monitoring of Kawasaki disease or MIS-C. The panel includes target-binding agents that bind at least two protein markers selected from Table 1. The panel optionally includes at least one clinical variable selected from Table 2.

### BRIEF DESCRIPTION OF THE DRAWINGS

FIG. 1 shows a receiver operating characteristic curve for an example KD panel (KDA001, as described in Table 3, Example 1) (N=150) to diagnose the Kawasaki disease and/or monitor Kawasaki disease progression or therapeutic effect. The panel had a robust cross-validated area under the curve (AUC) of 0.91 (data not shown), and an in-sample AUC of 0.91 (shown in FIG. 1).

FIG. 2 shows a receiver operating characteristic curve for an example KD panel (KDA030, as described in Table 3, Example 2) (N=150) to diagnose the Kawasaki disease and/or monitor Kawasaki disease progression or therapeutic effect. The panel had a robust cross-validated area under the curve (AUC) of 0.91 (data not shown), and an in-sample AUC of 0.92 (shown in FIG. 2).

FIG. 3 shows a receiver operating characteristic curve for an example KD panel (KDA080, as described in Table 3, Example 3) (N=150) to diagnose the Kawasaki disease and/or monitor Kawasaki disease progression or therapeutic effect. The panel had a robust cross-validated area under the curve (AUC) of 0.90 (data not shown), and an in-sample AUC of 0.91 (shown in FIG. 3).

FIG. 4 shows a receiver operating characteristic curve for an example KD panel (KDA038, as described in Table 3, Example 4) (N=150) to diagnose the Kawasaki disease and/or monitor Kawasaki disease progression or therapeutic effect. The panel had a robust cross-validated area under the curve (AUC) of 0.95 (data not shown), and an in-sample AUC of 0.95 (shown in FIG. 4).

FIG. 5 shows a receiver operating characteristic curve for an example KD panel (KDA089, as described in Table 3, Example 5) (N=150) to diagnose the Kawasaki disease and/or monitor Kawasaki disease progression or therapeutic effect. The panel had a robust cross-validated area under the curve (AUC) of 0.95 (data not shown), and an in-sample AUC of 0.95 (shown in FIG. 5).

FIG. 6 shows a receiver operating characteristic curve for an example KD panel (KDA017, as described in Table 3, Example 6) (N=150) to diagnose the Kawasaki disease and/or monitor Kawasaki disease progression or therapeutic effect. The panel had a robust cross-validated area under the curve (AUC) of 0.94 (data not shown), and an in-sample AUC of 0.94 (shown in FIG. 6).

FIG. 7 shows a receiver operating characteristic curve for an example KD panel (KDA086, as described in Table 3, Example 7) (N=150) to diagnose the Kawasaki disease and/or monitor Kawasaki disease progression or therapeutic effect. The panel had a robust cross-validated area under the curve (AUC) of 0.94 (data not shown), and an in-sample AUC of 0.95 (shown in FIG. 7).

FIG. 8 shows a receiver operating characteristic curve for an example KD panel (KDA012, as described in Table 3, Example 8) (N=150) to diagnose the Kawasaki disease and/or monitor Kawasaki disease progression or therapeutic effect. The panel had a robust cross-validated area under the curve (AUC) of 0.91 (data not shown), and an in-sample AUC of 0.92 (shown in FIG. 8).

FIG. 9 shows a receiver operating characteristic curve for an example KD panel (KDA085, as described in Table 3, Example 9) (N=150) to diagnose the Kawasaki disease and/or monitor Kawasaki disease progression or therapeutic effect. The panel had a robust cross-validated area under the curve (AUC) of 0.91 (data not shown), and an in-sample AUC of 0.92 (shown in FIG. 9).

FIG. 10 shows a receiver operating characteristic curve for an example KD panel (KDA043, as described in Table 3, Example 10) (N=150) to diagnose the Kawasaki disease and/or monitor Kawasaki disease progression or therapeutic effect. The panel had a robust cross-validated area under the curve (AUC) of 0.95 (data not shown), and an in-sample AUC of 0.96 (shown in FIG. 10).

FIG. 11 shows a receiver operating characteristic curve for an example KD panel (KDAU007, as described in Table 3, Example 10) (N=150) to diagnose the Kawasaki disease and/or monitor Kawasaki disease progression or therapeutic effect. The panel had a robust cross-validated area under the curve (AUC) of 0.91 (data not shown), and an in-sample AUC of 0.92 (shown in FIG. 11).

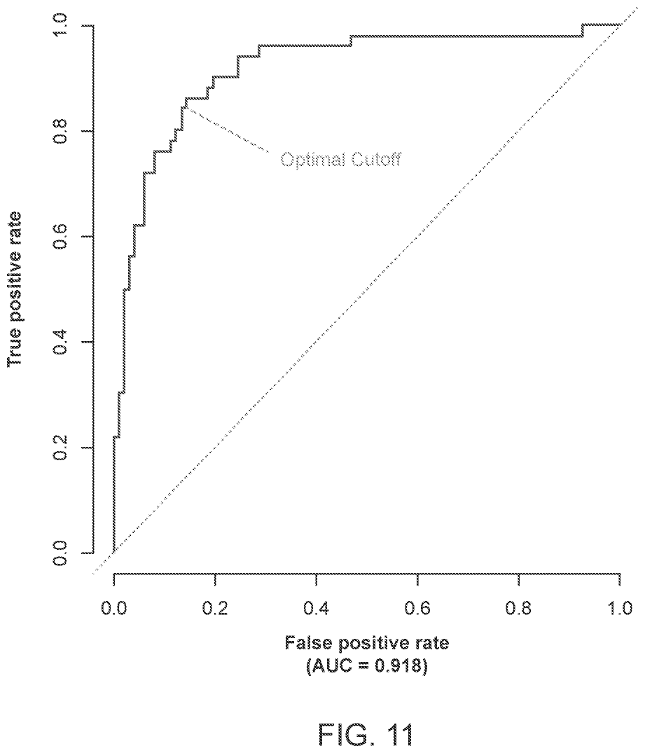

FIG. 12 presents a flow chart for the evaluation of suspected incomplete Kawasaki disease. In the absence of a “gold standard” for diagnosis, this algorithm represents the informed opinion of the American Heart Association expert committee on Kawasaki disease. Should a child only have two to three compatible clinical criteria (as opposed to four out of the five clinical criteria for complete Kawasaki disease), then another diagnosis should be considered including exudative conjunctivitis, exudative pharyngitis, ulcerative intraoral lesions, bullous or vesicular rash, generalized adenopathy, or splenomegaly. An infant with a fever for seven or greater days, without other clinical criteria for Kawasaki disease, are at particularly high risk of developing coronary artery abnormalities. Echocardiography is considered positive for purposes of this algorithm if any one of the following three conditions are met: 1) Z score of left anterior descending coronary artery or right coronary artery ≥2.5; 2) coronary artery aneurysm is observed; or 3) three or more other suggestive features exist, including decreased left ventricular function, mitral regurgitation, pericardial effusion, or Z scores in left anterior descending coronary artery or right coronary artery of 2 to 2.5. Should the echocardiogram positive, be as defined above, treatment should be given within 10 days of fever onset or after the tenth day of fever in the presence of clinical and laboratory signs of ongoing inflammation. Examples of laboratory signs include ≥3.0 mg/D1 C-reactive protein [CRP] and/or >40 mm/hr erythrocyte sedimentation rate [ESR].

### DETAILED DESCRIPTION

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art.

All patents, patent applications, articles and publications mentioned herein, both supra and infra, are hereby expressly incorporated herein by reference in their entireties.

### Definitions

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment described herein. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2, or 1% to a reference quantity, level, value, concentration, measurement, number, frequency, percentage, dimension, size, amount, weight or length. In particular embodiments, the terms “about” or “approximately” when preceding a numerical value indicates the value plus or minus a range of 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The terms “disease” or “condition” refer to a state of being or health status of a patient or subject capable of being treated with the compounds or methods provided herein. The disease may be an autoimmune disease. The disease may be an inflammatory disease. The disease may be an infectious disease. The disease may be Kawasaki disease.

As used herein, the term “diagnosis” refers to an identification or likelihood of the presence of Kawasaki disease or outcome in a subject. As also used herein, the term “prognosis” refers to the likelihood or risk of a subject developing a particular outcome or particular event. The phrase “determining Kawasaki Disease” or “determining MIS-C” may include the process of obtaining a diagnosis, prognosis, and monitoring of the disease status in a subject.

As used herein, a “biological sample” encompasses essentially any sample type that can be used in a diagnostic or prognostic method described herein. The biological sample may be any bodily fluid, tissue or any other sample from which clinically relevant protein marker levels may be determined. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polypeptides or proteins. The term “biological sample” encompasses a clinical sample, but also, in some instances, includes cells in culture, cell supernatants, cell lysates, blood, serum, plasma, urine, cerebral spinal fluid, biological fluid, and tissue samples. The sample may be pretreated as necessary by dilution in an appropriate buffer solution or concentrated, if desired. Any of a number of standard aqueous buffer solutions, employing one of a variety of buffers, such as phosphate, Tris, or the like, preferably at physiological pH can be used.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters including the results of a physical examination, neuropsychiatric exams, and/or a psychiatric evaluation. The term “treating” and conjugations thereof may include prevention of an injury, pathology, condition, or disease. In embodiments, treating is preventing. In embodiments, treating does not include preventing.

“Treating” or “treatment” as used herein (and as well understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. As such, “treatment” as used herein includes therapeutic treatment. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the risk or condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

An “effective amount” is an amount sufficient for a composition to accomplish a stated purpose relative to the absence of the composition (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of the drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “prevent” refers to a decrease in the occurrence of disease symptoms in a patient. The prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition. Non-limiting examples include humans, other mammals, bovines, rats, mice, dogs, monkeys, goat, sheep, cows, deer, and other non-mammalian animals. In some embodiments, a patient is human.

“Control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). In some instances, the control is a quantification standard used as a reference for assay measurements. The quantification standard may be a synthetic protein marker, a recombinantly expressed purified protein marker, a purified protein marker isolated from its natural environment, a protein fragment, a synthesized polypeptide, or the like.

The term “coronary artery” refers to one or more of arterial blood vessels of coronary circulation, which transport oxygenated blood to the heart muscle. The heart requires a continuous supply of oxygen to function and survive, much like any other tissue or organ of the body. The coronary arteries wrap around the entire heart. The two main branches are the left coronary artery (LC A) and right coronary artery (RCA). Reduced function of the coronary arteries can lead to decreased flow of oxygen and nutrients to the heart. Not only does this affect supply to the heart muscle itself, but it also can affect the ability of the heart to pump blood throughout the body. Therefore, any disorder or disease of the coronary arteries can have a serious impact on health, possibly leading to angina, a heart attack, and even death.

The term “Kawasaki disease” refers to acute vasculitis that predominantly affects young children. The symptoms of Kawasaki disease include but is not limited to the presence of a persistent fever for ≥3 days, and the presence of at least 1 or more of the following clinical features: cervical lymphadenopathy, erythema and edema of the feet and hands, periungual desquamation, a rash including maculopapular, diffuse erythroderma, or erythema multiforme-like, bilateral bulbar conjunctival injection without exudate, and erythema and cracking of lips, strawberry tongue, and/or erythema of oral and pharyngeal mucosa. Symptoms can also include: myocarditis, pericarditis, valvular regurgitation, shock, coronary artery abnormalities, aneurysms of medium-size noncoronary arteries, peripheral gangrene, aortic root enlargement, peribronchial and intestinal infiltrates on chest x-ray, pulmonary nodules, arthritis, arthralgia (pleocytosis of synovial fluid), diarrhea, vomiting, abdominal pain, hepatitis, jaundice, gallbladder hydrops, pancreatitis, extreme irritability, aseptic meningitis (pleocytosis of synovial fluid), facial nerve palsy, urethritis/meatitis, hydrocele, desquamating rash in groin, retropharyngeal phlegmon, anterior uveitis, and erythema and induration at BCG inoculation site.

The terms “incomplete Kawasaki disease” or “atypical Kawasaki disease” refer to a condition where the patient displays a fever for ≥5 days, and 2 or 3 compatible clinical criteria (as opposed to four out of the five clinical criteria for complete Kawasaki disease), Compatible clinical criteria include: 1) erythema and cracking of lips, strawberry tongue and/or erythema of the oral pharyngeal mucosa; 2) bilateral bulbar conjunctival injection without exudate; 3) rash: maculopapular, diffuse erythroderma or erythema multiforme-like; 4) erythema and edema of the hands and feet in acute phase and/or periungual desquamation in subacute phase; 5) cervical lymphadenopathy (≥1.5 cm diameter), usually unilateral.

The term “cardiovascular event” as used herein denotes a variety of adverse outcomes related to the cardiovascular system.

As described herein, the terms “marker”, “protein marker”, “polypeptide marker,” and “biomarker” are used interchangeably throughout the disclosure. As used herein, a protein marker refers generally to a protein or polypeptide, the level or concentration of which is associated with a particular biological state, particularly a state associated with a Kawasaki disease, event or outcome. Panels, assays, kits and methods described herein may comprise antibodies, binding fragments thereof or other types of target-binding agents, which are specific for the protein marker described herein.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. In various embodiments, detecting the levels of naturally occurring protein marker proteins in a biological sample is contemplated for use within diagnostic, prognostic, or monitoring methods disclosed herein. The term also includes fusion proteins, including, but not limited to, naturally occurring fusion proteins with a heterologous amino acid sequence, fusions with heterologous and homologous leader sequences, with or without N-terminal methionine residues; immunologically tagged proteins; and the like. The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “antibody” herein is used in the broadest sense and specifically covers, but is not limited to, monoclonal antibodies, polyclonal antibodies, multi-specific antibodies (e.g., bispecific antibodies) formed from at least two intact antibodies, single chain antibodies (e.g., scFv), and antibody fragments or other derivatives, so long as they exhibit the desired biological specificity. The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “monoclonal antibody” as used herein refers to an antibody obtained from a population of substantially homogeneous antibodies, i.e., the individual antibodies comprising the population are identical except for possible naturally occurring mutations that can be present in minor amounts. In certain specific embodiments, the monoclonal antibody is an antibody specific for a protein marker described herein.

Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity and/or specificity. Methods of making chimeric antibodies are known in the art.

An “isolated” antibody is one that has been identified and separated and/or recovered from a component of its natural environment. Contaminant components of its natural environment are materials that would interfere with diagnostic or prognostic uses for the antibody, and may include enzymes, hormones, and other proteinaceous or non-proteinaceous solutes. In specific embodiments, the antibody will be purified to greater than 95% by weight of antibody, e.g., as determined by the Lowry method, and most preferably more than 99% by weight.

The terms “detectably labeled antibody” refers to an antibody (or antibody fragment) which retains binding specificity for a protein marker described herein, and which has an attached detectable label. The detectable label can be attached by any suitable means, e.g., by chemical conjugation or genetic engineering techniques. Methods for production of detectably labeled proteins are well known in the art. Detectable labels may be selected from a variety of such labels known in the art, including, but not limited to, haptens, radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, and luciferase/luciferin)), methods for labeling antibodies, and methods for using labeled antibodies are well known in the art.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, immunoassays are routinely used to select antibodies specifically immunoreactive with a protein.

An example immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “VH,” or “VH” refer to the variable region of an immunoglobulin heavy chain, including an Fv, scFv, dsFv or Fab, while the terms “variable light chain,” “VL” or “VL” refer to the variable region of an immunoglobulin light chain, including of an Fv, scFv, dsFv or Fab.

“Functional fragments” of antibodies can also be used and include those fragments that retain sufficient binding affinity and specificity for a protein marker to permit a determination of the level of the protein marker in a biological sample. In some cases, a functional fragment will bind to a protein marker with substantially the same affinity and/or specificity as an intact full chain molecule from which it may have been derived. Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen. As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use as described herein include humanized and/or chimeric monoclonal antibodies.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form.

A “substantially isolated” or “isolated” substance is one that is substantially free of its associated surrounding materials in nature. By substantially free is meant at least 50%, preferably at least 70%, more preferably at least 80%, and even more preferably at least 90% free of the materials with which it is associated in nature. As used herein, “isolated” can refer to polynucleotides, polypeptides, antibodies, cells, samples, and the like.

As used herein, “alpha-1 antitrypsin” or “α₁-antitrypsin” refers to a protein involved with inhibiting serine proteases, especially enzymes secreted, or released by inflammatory cells, including neutrophil elastase and trypsin, and is encoded by the SERPN1A1 gene. It is also referred to as A1AT, α₁AT, A1A, AAT, α₁-PI, and historically, serum trypsin inhibitor (STI).

As used herein, “alpha-1 microglobulin” or “α₁-microglobulin” refers to a protein involved with scavenging free radicals, binding and degrading free heme groups, acts as a reductase because of its free surface cysteine, and is encoded by the AMBP gene. It is also referred to as A1M and protein HC.

As used herein, “angiopoietin 1” or “ANG-1” refers to a type of angiopoietin and is encoded by the gene ANGPT1. Angiopoietins are proteins with important roles in vascular development and angiogenesis. All angiopoietins bind with similar affinity to an endothelial cell-specific tyrosine-protein kinase receptor. The protein encoded by this gene is a secreted glycoprotein that activates the receptor by inducing its tyrosine phosphorylation. It plays a critical role in mediating reciprocal interactions between the endothelium and surrounding matrix and mesenchyme. The protein also contributes to blood vessel maturation and stability, and may be involved in early development of the heart.

As used herein, “apolipoprotein(a)”, also referred to as “apo(a)”, is the main constituent of lipoprotein(a) (Lp(a)). Apolipoprotein(a) has serine proteinase activity and is capable of auto proteolysis. Apolipoprotein(a) inhibits tissue-type plasminogen activator 1. Apolipoprotein(a) is known to be proteolytically cleaved, leading to the formation of the so-called mini-Lp(a). Apolipoprotein(a) fragments accumulate in atherosclerotic lesions, where they may promote thrombogenesis.

As used herein, “beta-2-microglobulin” or “β2-globulin”, is a protein that, along with the α-chain, forms the extracellular component of major histocompatibility complex I (MHCI). Additional functions include associating the MHCI-like molecules and the HFE protein, in order to regulate the expression of hepcidin. It is also referred to as B2M and is encoded by the B2M gene.

As used herein, “brain-derived neurotropic factor” or “BDNF”, is a protein, which functions include supporting survival, growth and differentiation of neurons, neurogenesis, and long-term memory formation. It is also known as abreneurin and is encoded by the BDNF gene.

As used herein, “CD163” or “cluster of differentiation 163”, is a high affinity scavenger receptor for hemoglobin/haptoglobin complex, and a low affinity scavenger of hemoglobin alone, an innate immune sensor for bacterial infection, and a marker for monocytes/macrophages. CD163 is encoded by the CD163 gene, exists both as a membrane bound cell surface protein and as a circulating soluble version.

As used herein, “CD5 Antigen-like” is a secreted protein that regulates lipid synthesis, expressed mainly by macrophages. The protein is encoded by the CD5L gene.

As used herein, “clusterin” is a protein that acts as an extracellular chaperone, preventing aggregation, especially important for preventing stress-induced aggregation of blood proteins. The protein is encoded by the CLU gene.

As used herein, “Complement C3” or “C3” is the central, most abundant protein in both the classical, alternative, and lectin complement pathways of innate immunity. C3 is processed by the C3 convertase into C3a and C3b in the classical and lectin pathways. C3b is an opsonizing agent for pathogens, immune complexes, and apoptotic cells. Alternatively, C3b can be converted into C3bBb by association by Factor Bb, which becomes a C3 convertase on the surfaces of pathogens after associating with properdin (Factor P). An additional C3b added to C3bB generates C5 convertase, which amplifies the complement signals by processing many more C3 molecules. The protein C3 is encoded by the gene C3.

As used herein, “C-reactive protein” or “CRP” is an acute-phase reactant protein that responds rapidly to inflammation.

As used herein, “Cystatin-C,” “cystatin 3”, “gamma trace, post-gamma-globulin”, and “neuroendocrine basic polypeptide” is a protein encoded by the CST3 gene, is mainly used as a biomarker of kidney function. Recently, it has been studied for its role in predicting new-onset or deteriorating cardiovascular disease. In humans, all cells with a nucleus (cell core containing the DNA) produce cystatin C as a chain of 120 amino acids. It is found in virtually all tissues and body fluids. It is a potent inhibitor of lysosomal proteinases and an important extracellular inhibitor of cysteine proteases (it prevents the breakdown of proteins outside the cell by a specific type of protein degrading enzymes). Cystatin C belongs to the type 2 cystatin gene family.

As used herein, “Eotaxin-1” or “C—C motif chemokine 11” or “eosinophil chemotactic protein” is a small protein belonging to the CC chemokine family, which functions to specifically attract eosinophils and is proposed to be involved with allergic responses.

As used herein “Factor VII”, also known as “blood-coagulation factor VIIa”, “activated blood coagulation factor VII”, or “proconvertin” is one of the proteins that causes blood to clot in the coagulation cascade. It is an enzyme of the serine protease class.

As used herein, “fibrinogen” or “factor 1” is a glycoprotein complex that circulates in the blood. Fibrinogen is a complex of Aα, Bβ, and γ fibers that are eventually assembled into a (AαBβγ)2 heximer, which is linked via multiple disulfides.

As used herein, “growth/differentiation factor 15” or “macrophage inhibitory cytokine-1 (MIC-1)” refers to a protein that is upregulated due to injury of organs including the lungs, heart, liver, and kidney. The protein is encoded by the GDF15 gene.

As used herein, “haptoglobin” or “Hp” is a protein that binds free hemoglobin in the plasma, which allows for degradation of hemoglobin without the loss of iron in the kidneys. Levels of haptoglobin can be increased due to an acute infection or inflammatory response. The protein is encoded by the HP gene.

As used herein “immunoglobulin A” or “IgA” is an antibody that plays a crucial role in the immune function of mucous membranes. The amount of IgA produced in association with mucosal membranes is greater than all other types of antibody combined. IgA has two subclasses (IgA1 and IgA2) and can be produced as a monomeric as well as a dimeric form. The IgA dimeric form is the most prevalent and is also called secretory ISA (sIgA). Both IgA1 and IgA2 are heavily glycosylated proteins. While IgA1 predominates in serum (˜80%), IgA2 percentages are higher in secretions than in serum (˜35% in secretions).

As used herein “immunoglobulin M” or “IgM” is one of several forms of antibody that are produced by vertebrates. IgM is the largest antibody, and it is the first antibody to appear in the response to initial exposure to an antigen.

As used herein “Intercellular Adhesion Molecule 1” also known as “ICAM-1” and “CD54” or “Cluster of Differentiation 54” is a cell surface glycoprotein, which is typically expressed on endothelial cells and cells of the immune system. It binds to integrins of type CD11a/CD18, or CD11b/CD18.

As used herein, “interleukin-1 alpha” or “IL-1α” or “IL-1α” or “hematopoetin-1” is a proinflammatory cytokine belonging to the interleukin-1 family of proteins. Historically, IL-1α is known as “lymphocyte-activating factor (LAF)”, along with IL-1(3. IL-1α is a dual function cytokine: the N-terminal region contains a nuclear localization sequence and binds DNA, and the C-terminal region can form a complex with the interleukin-1 receptor, type 1 (IL-1R1), to initiate cell signaling cascades critical for innate immune responses and wound healing. IL-1α is constitutively expressed by epithelial cells, and transiently expressed by most immune cells, fibroblasts, endothelial cells, maternal placental cells, and kidney mesangial cells, among other cells. The protein is encoded by the ILIA gene.

As used herein, “interleukin-1 beta” or “IL-1(3” or “IL-1b” is a proinflammatory cytokine of the interleukin-1 family of cytokines. IL-1β is a tightly-regulated, non-classically secreted, transiently expressed, extremely potent cytokine expressed as a much longer, inactive precursor sequence (Pro-IL-1β). Pro-IL-1β is canonically processed by caspase-1, and the mature C-terminal region folds and binds interleukin-1 receptor type 1 (IL-1R1), which initiates numerous intracellular signaling cascades. IL-1β is mainly expressed by monocytes and macrophages, and related cell types, and is a master innate immune system modulator, important for wound healing, cell proliferation/differentiation, cancer progression (gastric), atherosclerosis, sleep regulation, and central nervous system function. The protein is encoded by the IL1B gene.

As used herein, “interleukin-1 receptor antagonist” or “IL-1Ra” also known as “interleukin-1 inhibitor” or “IL-1 inhibitor”, refers to a protein that is a member of the interleukin-1 cytokine family Historically, IL-1Ra is known as “interleukin-1 receptor antagonist protein (IRAP)”. IL-1Ra is secreted by various types of cells including immune cells, epithelial cells, and adipocytes, and is a competitive inhibitor of proinflammatory cytokines IL-1α and IL-1β binding to interleukin-1 receptor type 1 (IL-IR1). IL-1 Ra is encoded by IL1RN.

As used herein, “interleukin-12 subunit p′70” or “IL-12 p′70” or “IL-12” is an extracellular cytokine, comprising a heterodimer that is formed by the association of interleukin-12 p35 (IL-12A) and interleukin-12 p40 (IL-12B). IL-12 is expressed by immune cells including B-cells, macrophages, neutrophils, and dendritic cells, among others, and is involved with modulating both adaptive and innate immune responses. It is encoded by both the IL12A and IL12B genes.

As used herein, “interleukin-12 subunit p40” or “IL-12 p40” or “IL-12B” is a subunit of the extracellular cytokine IL-12. When combined with IL-12A, it forms IL-12. When combined with IL-23A, it forms IL-23. The protein is encoded by the IL12B gene.

As used herein, “interleukin-17” or “IL-17” or “CTL8A” refers to a protein that acts as a proinflammatory cytokine, and is produced in Thu polarized helper T-cells. IL-17 assists in IL-1 and TNF-mediated immune responses. Several other isoforms of IL-17 exist as IL-17B, IL-17C, IL-17D, IL-17E, and IL-17F. IL-17A is encoded by the IL17A gene.

As used herein, “kidney injury molecule 1”, also known as “kidney injury molecule-1” and “KIM-1” is a type I cell membrane glycoprotein that serves as a receptor for oxidized lipoproteins and plays a functional role in the kidney. KIM-1 is a proximal renal tubular marker, concentrations of which have been linked to acute kidney injury.

As used herein, “matrix metalloproteinase-3” also known as “MMP-3” or “Stromelysin-1” is an enzyme encoded by the MMP3 gene. MMP-3 is a metalloenzyme that is mainly expressed by smooth muscle cells, and its function is to degrade the extracellular matrix and to participate in wound healing and connective tissue remodeling.

As used herein, “matrix metalloproteinase-9”, also known as “MMP-9”, “92 kDa type IV collagenase”, “92 kDa gelatinase”, and “gelatinase B” or “GELB”, is a matrix in a class of enzymes that belong to the zinc-metalloproteinase family involved in the degradation of the extracellular matrix. Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis.

As used herein, “midkine”, also known as “neurite growth-promoting factor 2” or “NEGF2”, refers to a basic heparin-binding growth factor of low molecular weight and forms a family with pleiotrophin. Midkine is a heparin-binding cytokine/growth factor with a molecular weight of 13 kDa.

As used herein, “N-terminal prohormone of brain natriuretic peptide” or “NT-PBNP” is also known as “NT-proBNP” or “BNPT” and refers to an N-terminal inactive protein that is cleaved from proBNP to release brain natriuretic peptide.

As used herein, “ostercalcin” or “bone gammacarboxyglutamic acid-containing protein” or “BGLAP” is a small protein found in bone and dentin in teeth, and is a biomarker for bone formation. It is also released during an acute stress response.

As used herein, “osteopontin”, also known as “bone sialoprotein 1”, “BSP-1”, “BNSP”, “early T-lymphocyte activation”, “ETA-1”, “secreted phosphoprotein 1”, “SPP1”, “2ar”, “Rickettsia resistance”, or “Ric”, refers to a glycoprotein (small integrin binding ligand N-linked glycoprotein) first identified in osteoblasts. It includes all isoforms and post-translational modifications.

As used herein, “periostin” or “osteoblast-specific factor OSF-2” refers to a protein that is a secreted extracellular matrix protein. OSF-2 helps modulate tissue-remodeling and tissue-development processes. Misregulation of OSF-2 arises in cancer, asthma, and cardia valve degeneration.

As used herein, “p-selectin” or “SELP” refers to a protein that functions as a cell-adhesion molecule, especially on the inner surfaces of blood vessels. P-selectin is found in platelets, endothelial cells, and recruits leukocytes to the site of injury during an inflammatory response.

As used herein, “serum amyloid p-component” or “SAP” or “PTX2” refers to protein that belongs to the pentraxin family of proteins, and is found in amyloid deposits, in amyloidosis (amyloid disease). SAP acts as a protein that can selectively inhibit fibril formation, and can also help modulate immune responses from macrophages and neutrophils.

As used herein, “ST2”, or “T1/ST2” or “interleukin-1 receptor-like 1” refers to a protein that is an extracellular receptor for the cytokine interleukin-33, and belongs to the IL-1 family of proteins. ST2 is expressed as both a soluble N-terminal region consisting for 3 Ig-containing cytokine binding protein (sST2), or as a full-length cell surface receptor containing the extracellular facing cytokine binding domain, a transmembrane helix, and an intracellular TIR domain (ST2L). sST2 is upregulated in instances in mechanical tissue damage. ST2L has been shown to interact with c-KIT, and is expressed in barrier organs, especially the lungs, and by T-cells and cardiomyocytes.

As used herein, “stem cell factor”, also known as SCF, KIT-ligand, KL, and steel factor, is a cytokine that binds to the c-KIT receptor (CD117). SCF can exist as both a transmembrane protein and a soluble protein. This cytokine plays an important role in hematopoiesis (formation of blood cells), spermatogenesis, and melanogenesis.

As used herein, “Thyroxine Binding Globulin” or “TBG” a globulin that binds thyroid hormones in circulation. It is one of three transport proteins (along with transthyretin and serum albumin) responsible for carrying the thyroid hormones thyroxine (T₄) and triiodothyronine (T₃) in the bloodstream.

As used herein, “vascular endothelial growth factor,” “VEGF,” or “vascular permeability factor”, or “VPF”, is a signal protein produced by cells that stimulates the formation of blood vessels. To be specific, VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature).

As used herein, “von Willebrand Factor” or “vWF” is a blood glycoprotein involved in hemostasis. Its primary function is binding to other proteins, in particular factor VIII, cells, and molecules. It is important in platelet adhesion to wound sites, thus playing a major role in blood coagulation. It is not an enzyme and, thus, has no catalytic activity.

As used herein, “thyroid stimulating hormone (TSH)” is a pituitary hormone that stimulates the thyroid gland to produce thyroxine (T4) and then triiodothyronine (T3), which stimulates the metabolism of almost every tissue in the body. It is a glycoprotein hormone produced by thyrotrope cells in the anterior pituitary gland, which regulates the endocrine function of the thyroid.

As used herein, “thyroxine (T4)” is a circulating thyroid hormone that is produced by the follicular cells of the thyroid gland. It is converted in the hypothalamus and pituitary to another thyroid hormone, triiodothyronine (T3), by the 5′-deiodinase type 2.

As used herein, “triiodothyronine (T3)” is a thyroid hormone whose production is activated by TSH, which is released from the anterior pituitary gland. T3 affects every physiological process in the body, including growth and development, metabolism, body temperature, and heart rate.

As used herein, “B-type natriuretic peptide (BNP)” is a hormone secreted by cardiomyocytes in the heart ventricles in response to stretching caused by increased ventricular blood volume. BNP binds to and activates the atrial natriuretic factor receptor NPRA, and to a lesser extent NPRB, in a fashion similar to atrial natriuretic peptide (ANP) but with 10-fold lower affinity. The physiological actions of BNP include decrease in systemic vascular resistance and central venous pressure as well as an increase in natriuresis. The net effect of these peptides is a decrease in blood pressure due to the decrease in systemic vascular resistance and, thus, afterload. Additionally, the actions of both BNP and ANP result in a decrease in cardiac output due to an overall decrease in central venous pressure and preload as a result of the reduction in blood volume that follows natriuresis and diuresis.

As used herein, “T Uptake” refers to thyroid hormone uptake (“T3 uptake” or “T4 uptake”) and is a measure of the unbound thyroxine binding proteins in the blood, that is, the thyroid binding protein that is unsaturated with thyroid hormone. Unsaturated thyroid binding protein increases with decreased levels of thyroid hormones.

As used herein, “Vitamin D-Binding Protein” refers to a multifunctional protein found in plasma, ascetic fluid, cerebrospinal fluid and on the surface of many cell types. It is able to bind the various forms of vitamin D including ergocalciferol (vitamin D2) and cholecalciferol (vitamin D3), the 25-hydroxylated forms (calcifediol), and the active hormonal product, 1,25-dihydroxyvitamin D (calcitriol). The major proportion of vitamin D in blood is bound to this protein. It transports vitamin D metabolites between skin, liver and kidney, and then on to the various target tissues.

It will be understood by one skilled in the art that these and other protein markers disclosed herein (e.g., those set forth in Table 1) can be readily identified, made and used in the context of the present disclosure in light of the information provided herein.

As used herein, the term “score” refers to a binary, tertiary, multilevel, or continuous result as it relates diagnostic or prognostic determinations. A score can be a positive or negative diagnostic score. A score can be a positive, intermediate, or negative diagnostic score. A score can be a positive or negative prognostic score. A score can be a positive, intermediate, or negative prognostic score.

As used herein, the term “panel” refers to specific combination of protein markers and clinical markers used to determine a diagnosis or prognosis of Kawasaki disease or outcome in a subject. The term “panel” may also refer to an assay comprising a set of protein markers used to determine a diagnosis or prognosis of Kawasaki disease or outcome in a subject.

As further described herein, the “training set” is the set of patients or patient samples that are used in the process of training (i.e., developing, evaluating and building) the final diagnostic or prognostic model. The “validation set” is a set of patients or patient samples that are withheld from the training process and are only used to validate the performance of the final diagnostic or prognostic model. If the set of patients or patient samples are limited in number, all available data may be used as a training set, or as an “in-sample” validation set.

As used herein, the term “normalized” refers to a mathematical process applied to a numerical value, regardless of the input or output value. It may include taking protein concentrations and calculating the base-10 logarithm from original values, reflecting a “log transformation.”

protein MARKERS

Certain illustrative protein markers provided herein can be found listed in Table 1. Certain illustrative clinical variables provided herein can be found listed in Table 2. Based on the information therein, the skilled artisan can readily identify, select and implement a protein marker or protein marker combination, and, optionally, a clinical variable or clinical variable combination in accordance with the methods provided herein.

In embodiments, at least 2, at least 3, at least 4, at least 5, or at least 6 protein markers from Table 1 are used in accordance with the present disclosure. In an embodiment, two proteins from Table 1 are selected. In an embodiment, three proteins from Table 1 are selected. In an embodiment, four proteins from Table 1 are selected. In an embodiment, five proteins from Table 1 are selected. In an embodiment, six proteins from Table 1 are selected. In other embodiments, the number of protein markers employed can vary, and may include at least 7, 8, 9, 10, or more.

In embodiments, one, at least two, or at least three clinical variables from Table 2 are used in the methods and panels provided herein. In an embodiment, one clinical variable from Table 2 is selected. In an embodiment, two clinical variables from Table 2 are selected. In an embodiment, three clinical variables from Table 2 are selected. In an embodiment, four clinical variables from Table 2 are selected. In an embodiment, five clinical variables from Table 2 are selected. In other embodiments, the number of clinical variables employed can vary, and may include at least two, three, four, or more. In embodiments where MIS-C is determined, one or more clinical variables may also include suspicion of having MIS-C and/or SARS-CoV-2, and/or diagnosis of infection with SARS-CoV-2.

In embodiments, treatment of a subject comprises assigning a result. In embodiments, the result is positive, intermediate, or negative. In embodiments, the treatment comprises a therapeutic intervention regimen.

In embodiments, the biological sample is blood. In embodiments, the biological sample is plasma. In embodiments, the biological sample is serum.

In embodiments, the protein markers used in herein are selected from alpha-1 antitrypsin, C Reactive Protein, interleukin-1 beta, matrix metalloproteinase-9, N-terminal prohormone of brain natriuretic peptide, periostin, thyroxine-binding globulin and T uptake.

In embodiments, at least two of the protein markers used in accordance with the present disclosure are selected from alpha-1 antitrypsin, C Reactive Protein, interleukin-1 beta, matrix metalloproteinase-9, N-terminal prohormone of brain natriuretic peptide, periostin, thyroxine-binding globulin and T uptake, and where the optional step includes determining the status of at least one clinical variable selected from age, fever equal to or greater than 38.1° C., and race.

In embodiments, at least two of the protein markers used in accordance with the present disclosure are selected from C Reactive Protein and N-terminal prohormone of brain natriuretic peptide.

In embodiments, at least two of the protein markers used in accordance with the present disclosure are selected from C Reactive Protein, N-terminal prohormone of brain natriuretic peptide, and periostin.

In embodiments, at least two of the protein markers used in accordance with the present disclosure are selected from alpha-1 antitrypsin, C Reactive Protein, and N-terminal prohormone of brain natriuretic peptide.

In embodiments, at least two of the protein markers used in accordance with the present disclosure are selected from C Reactive Protein, matrix metalloproteinase-9, and N-terminal prohormone of brain natriuretic peptide.

In embodiments, at least two of the protein markers used in accordance with the present disclosure are selected from alpha-1 antitrypsin, C Reactive Protein, matrix metalloproteinase-9, and N-terminal prohormone of brain natriuretic peptide.

In embodiments, at least two of the protein markers used in accordance with the present disclosure are selected from C Reactive Protein, interleukin-1 beta, and N-terminal prohormone of brain natriuretic peptide.

In embodiments, at least two of the protein markers used in accordance with the present disclosure are selected from alpha-1 antitrypsin, C Reactive Protein, interleukin-1 beta, and N-terminal prohormone of brain natriuretic peptide.

In embodiments, at least two of the protein markers used in accordance with the present disclosure are selected from C Reactive Protein, N-terminal prohormone of brain natriuretic peptide and thyroxine-binding globulin.

In embodiments, at least two of the protein markers used in accordance with the present disclosure are selected from alpha-1 antitrypsin, C Reactive Protein, N-terminal prohormone of brain natriuretic peptide and thyroxine-binding globulin.

In embodiments, at least two of the protein markers used in accordance with the present disclosure are selected from C Reactive Protein, matrix metalloproteinase-9, N-terminal prohormone of brain natriuretic peptide and thyroxine-binding globulin.

In embodiments, at least two of the protein markers used in accordance with the present disclosure are selected from C Reactive Protein, N-terminal prohormone of brain natriuretic peptide and T uptake (see Example 11).

In embodiments, one or more proteins as recited in Table 1 may be specifically excluded from any of the methods or panels described herein.

Table 1 is a list of 47 proteins whose levels are correlated to the diagnosis and/or monitoring of Kawasaki disease, atypical or incomplete Kawasaki disease, and related disorders.

TABLE 1
Biomarkers

| | | | | | |
|---|---|---|---|---|---|
| Alpha-1 | Alpha-1- | Ang-1 | Apolipoprotein(a) | Beta-2-Microglobulin | Brain-Derived |
| Antitrypsin | Microglobulin | | | | Neurotrophic |
| | | | | | Factor (BDNF) |
| B-type | CD163 | CD5 | Clusterin | Complement C3 | C-Reactive |
| natriuretic | | Antigen- | | | Protein |
| peptide (BNP) | | like | | | |
| Cystatin-C | Eotaxin-1 | Factor VII | Fibrinogen | Growth/ | Haptoglobin |
| | | | | differentiation | |
| | | | | factor 15 | |
| Immunoglobulin A | Immunoglobulin M | Intercellular | Interleukin-1 | Interleukin-1 | Interleukin-1 |
| | | Adhesion | alpha | beta | receptor |
| | | Molecule 1 | | | antagonist |
| Interleukin-12 | Interleukin-12 | Interleukin-17 | Kidney Injury | Matrix | Matrix |
| Subunit p40 | Subunit p70 | | Molecule-1 | Metalloproteinase-3 | Metalloproteinase-9 |
| | | | (KIM-1) | | |
| Midkine | N Terminal | Osteocalcin | Osteopontin | Periostin | P-Selectin |
| | pro-Brain | | | | |
| | Natriuretic | | | | |
| | Peptide | | | | |
| | (NT-proBNP) | | | | |
| Serum Amyloid | ST2 | Stem Cell | Thyroid | Thyroid Hormone | Thyroid- |
| P-Component | | Factor | Hormone Uptake | Uptake (T4 Uptake | stimulating |
| | | | (T3 Uptake or | or T Uptake) | hormone (TSH) |
| | | | T Uptake) | | |
| Thyroxine (T4) | Thyroxine- | Vascular | Vitamin D- | Von Willebrand | |
| | Binding | Endothelial | Binding Protein | Factor | |
| | Globulin | Growth Factor | | | |
| | (TBG) | | | | |

In embodiments, the combination of proteins whose concentrations are correlated to the diagnosis, prognosis, and/or monitoring of Kawasaki disease or incomplete Kawasaki disease, or MIS-C, and the nature of whether those protein concentrations are increased, decreased, or the same as compared to a healthy individual provides a subject's protein profile.

### Clinical Variables

In embodiments, the protein markers described herein can optionally be used in combination with certain clinical variables in order to provide for an improved diagnosis, prognosis and/or monitoring of Kawasaki disease or MIS-C in a subject. As used herein, “optionally” refers to inclusion based on combinations of protein markers and their predictive value of Kawasaki disease or MIS-C or outcome when combined with a clinical variable factor. For example, illustrative clinical variables useful in the context of the present disclosure can be found listed in Table 2.

In embodiments, at least 1, at least 2, at least 3 or at least 4 clinical variables from Table 2 are used in the methods and panels provided herein. In an embodiment, one clinical variable from Table 2 is selected. In an embodiment, two clinical variables from Table 2 are selected. In an embodiment, three clinical variables from Table 2 are selected. In an embodiment, four clinical variables from Table 2 are selected. In an embodiment, five clinical variables from Table 2 are selected. In an embodiment, a combination of clinical variables is selected from Table 2.

In embodiments, the clinical variable(s) used in accordance with the present disclosure are selected from age, race, body temperature ≥38.1° C., suspicion of having Kawasaki disease, or three (3) or more days of persistent fever. In embodiments, the clinical variable used in accordance with the present disclosure is age. In embodiments, the clinical variable used in accordance with the present disclosure is race. In embodiments, the clinical variable used in accordance with the present disclosure is body temperature ≥38.1° C. In embodiments, the clinical variable used in accordance with the present disclosure is suspicion of Kawasaki disease. In embodiments, the clinical variable used in accordance with the present disclosure is 3 or more days of persistent fever.

In embodiments, the presence/absence of clinical factors represented in binary form (e.g., race, suspicion of Kawasaki disease), and/or clinical factors in quantitative form (e.g., body temperature, age) provide values that are entered into the diagnostic or prognostics model provided by software, and the result is evaluated against one or more cutoffs to determine the diagnosis or prognosis of Kawasaki disease.

In embodiments, a clinical characteristic as recited in Table 2 may be specifically excluded from the methods described herein.

Table 2 is a list of clinical variables and lab measurements correlated to the diagnosis, prognosis, and/or monitoring of Kawasaki disease.

TABLE 2
Clinical Variables

Age
Race
Fever ≥ 38.1° C.
Suspicion of Kawasaki disease
Persistent fever for 3 or more days

### Score

As further described herein, the diagnostic score can be used in an improved diagnosis, prognosis and/or monitoring of Kawasaki disease or MIS-C in a subject. As used herein, “optionally” refers to possible inclusion of a given criterion, based on combinations of protein markers and their predictive value of Kawasaki disease or MIS-C or outcome when combined with a clinical variable factor. The score is calculated using an algorithm based on the transformed and normalized concentration of protein markers determined after measuring via an analytical device, and, optionally, the status of the clinical variable(s) as determined for the patient. In embodiments, the score is a diagnostic score. In embodiments, the score is a prognostic score.

In embodiments, the diagnostic score is given as a positive, intermediate, or negative score. The diagnostic score is used for determining whether a patient has Kawasaki disease or MIS-C. The diagnostic score may also determine the course of treatment or therapeutic regime for a patient.

A positive diagnostic score in the subject facilitates a determination by a medical practitioner of the need for one or more interventions selected from administration of pharmacological agents, echocardiography, and avoidance of high dose acetylsalicylic acid (ASA) in patients with concomitant active infection with varicella or influenza. Further, pharmacological agents are one or more of intravenous immunoglobulin G, acetylsalicylic Acid (ASA), methylprednisolone, and infliximab.

An intermediate diagnostic score in the subject facilitates a determination by a medical practitioner of the need for one or more interventions selected from further testing and monitoring.

A negative diagnostic score in the subject facilitates a determination by a medical practitioner of the need for one or more interventions selected from further testing, differential diagnosis of other diseases, including measles, other viral infections (e.g., adenovirus, enterovirus), and juvenile idiopathic arthritis or other conditions including drug hypersensitivity reactions, including Stevens Johnson syndrome.

### Methods

In an aspect, provided herein are methods of determining Kawasaki disease in a subject including (i) providing a biological sample from a subject suspected of having Kawasaki disease; (ii) applying the biological sample to an analytical device including (a) detecting a concentration of at least two protein markers in the biological sample; (b) calculating the concentration of the at least two protein markers against a synthetic quantification standard to produce a protein marker concentration; (c) log transforming the concentration of the at least two protein markers to conform to a normal distribution; and (d) normalizing the log-transformed concentrations of the at least two protein markers to an established range and scale, where the at least two protein markers are selected from those set forth in Table 1; (iii) optionally, determining the status of at least one clinical variable for the subject, where the clinical variable is selected from those set forth in Table 2; (iv) calculating a diagnostic score using an algorithm that applies different weightings to a transformed, normalized concentration of protein markers determined in step (ii) and, optionally, the status of the clinical variable(s) determined in step (iii); (v) classifying the diagnostic score as a positive, intermediate, or negative score; and (vi) determining Kawasaki disease in the subject as indicated by the diagnostic score.

In an aspect, provided herein is a method of administering a therapeutic intervention to a subject suspected of having Kawasaki disease including: (i) determining the subject's protein marker profile for a panel of protein markers including at least two protein markers selected from those set forth in Table 1; (ii) optionally, determining the status of at least one clinical variable for the subject, where the clinical variable is selected from those set forth in Table 2; (iii) assigning a score to the subject based on the measured protein marker profile, and optionally the clinical value status, where the score is classified as a positive, intermediate, or negative score, said score algorithmically-derived from mathematically transformed and normalized concentrations of protein markers in the subject's sample and optionally, the status of at least one clinical variable; and (iv) administering to the subject a therapeutic intervention based on the positive, intermediate, or negative score.

In an aspect, provided herein, is a method of detecting two or more protein markers in a subject that is suspected of having Kawasaki disease, the method including: (i) selecting a subject that is suspected of having Kawasaki disease; (ii) providing a biological sample from the subject; (iii) applying the biological sample to an analytical device; (iv) detecting a concentration of at least two protein markers from Table 1; (v) calculating a diagnostic score using an algorithm that applies different weightings to the concentration of protein markers as determined by the analytical device, and, optionally, the status of the clinical variable(s) determined by the analytical device; (vi) classifying the diagnostic score as a positive, intermediate, or negative score; and (vii) determining Kawasaki disease in a subject as indicated by the diagnostic score.

In an aspect, provided herein is a method for treating a patient with a fever or suspected of having Kawasaki disease with intervention and additional testing including (i) determining whether the patient suffers from Kawasaki disease by: obtaining or having obtained a biological sample from the patient; performing or having performed a biomarker assay on the biological sample wherein the biomarker is selected from alpha-1 antitrypsin, C Reactive Protein, interleukin-1 beta, matrix metalloproteinase-9, N-terminal prohormone of brain natriuretic peptide, periostin and thyroxine-binding globulin and T uptake; and calculating a diagnostic score based on a weighted level of biomarker; and (ii) if the patient has a positive diagnostic score, then performing one or more interventions selected from administration of pharmacological agents or antibody (e.g., IgG), echocardiography, and avoidance of high dose acetylsalicylic acid (ASA) in patients with concomitant active infection with varicella or influenza, or enrolling in a clinical trial; if the patient has an intermediate score, then the need for one or more interventions selected from ongoing monitoring and further testing; and if the patient has a negative score, the need for one or more interventions selected from further testing, differential diagnosis of other diseases, including measles, other viral infections (e.g., adenovirus, enterovirus), and juvenile idiopathic arthritis or other conditions including drug hypersensitivity reactions, including Stevens Johnson syndrome.

Multisystem inflammatory syndrome in children (MIS-C) has been associated with coronavirus disease 2019 (COVID-19) infections in children. MIS-C may present with Kawasaki disease-like features. MIS-C can cause inflammation, including in the heart, lungs, kidneys, brain, skin, eyes, or gastrointestinal organs. Diagnosis includes:
An individual aged <21 years presenting with fever (>38.0° C. for ≥24 hours, or report of subjective fever lasting ≥24 hours), laboratory evidence of inflammation (Including, but not limited to, one or more of the following: an elevated C-reactive protein (CRP), erythrocyte sedimentation rate (ESR), fibrinogen, procalcitonin, d-dimer, ferritin, lactic acid dehydrogenase (LDH), or interleukin 6 (IL-6), elevated neutrophils, reduced lymphocytes and low albumin), and evidence of clinically severe illness requiring hospitalization, with multisystem (>2) organ involvement (cardiac, renal, respiratory, hematologic, gastrointestinal, dermatologic or neurological); AND
No alternative plausible diagnoses; AND
Positive for current or recent SARS-CoV-2 infection by RT-PCR, serology, or antigen test; or exposure to a suspected or confirmed COVID-19 case within the 4 weeks prior to the onset of symptoms.

In an aspect, provided herein are methods of determining MISC-C in a subject including (i) providing a biological sample from a subject suspected of having MISC-C; (ii) applying the biological sample to an analytical device including (a) detecting a concentration of at least two protein markers in the biological sample; (b) calculating the concentration of the at least two protein markers against a synthetic quantification standard to produce a protein marker concentration; (c) log transforming the concentration of the at least two protein markers to conform to a normal distribution; and (d) normalizing the log-transformed concentrations of the at least two protein markers to an established range and scale, where the at least two protein markers are selected from those set forth in Table 1; (iii) optionally, determining the status of at least one clinical variable for the subject, where the clinical variable is selected from those set forth in Table 2; (iv) calculating a diagnostic score using an algorithm that applies different weightings to a transformed, normalized concentration of protein markers determined in step (ii) and, optionally, the status of the clinical variable(s) determined in step (iii); (v) classifying the diagnostic score as a positive, intermediate, or negative score; and (vi) determining MISC-C in the subject as indicated by the diagnostic score.

In an aspect, provided herein is a method of administering a therapeutic intervention to a subject suspected of having MISC-C including: (i) determining the subject's protein marker profile for a panel of protein markers including at least two protein markers selected from those set forth in Table 1; (ii) optionally, determining the status of at least one clinical variable for the subject, where the clinical variable is selected from those set forth in Table 2; (iii) assigning a score to the subject based on the measured protein marker profile, and optionally the clinical value status, where the score is classified as a positive, intermediate, or negative score, said score algorithmically-derived from mathematically transformed and normalized concentrations of protein markers in the subject's sample and optionally, the status of at least one clinical variable; and (iv) administering to the subject a therapeutic intervention based on the positive, intermediate, or negative score.

In an aspect, provided herein, is a method of detecting two or more protein markers in a subject that is suspected of having MISC-C, the method including: (i) selecting a subject that is suspected of having MISC-C; (ii) providing a biological sample from the subject; (iii) applying the biological sample to an analytical device; (iv) detecting a concentration of at least two protein markers from Table 1; (v) calculating a diagnostic score using an algorithm that applies different weightings to the concentration of protein markers as determined by the analytical device, and, optionally, the status of the clinical variable(s) determined by the analytical device; (vi) classifying the diagnostic score as a positive, intermediate, or negative score; and (vii) determining MISC-C in a subject as indicated by the diagnostic score.

In an aspect, provided herein is a method for treating a patient with a fever or suspected of having MISC-C with intervention and additional testing including (i) determining whether the patient suffers from MISC-C by: obtaining or having obtained a biological sample from the patient; performing or having performed a biomarker assay on the biological sample wherein the biomarker is selected from alpha-1 antitrypsin, C Reactive Protein, interleukin-1 beta, matrix metalloproteinase-9, N-terminal prohormone of brain natriuretic peptide, periostin and thyroxine-binding globulin and T uptake; and calculating a diagnostic score based on a weighted level of biomarker; and (ii) if the patient has a positive diagnostic score, then performing one or more interventions selected from administration of pharmacological agents or antibody (e.g., IgG), echocardiography, and avoidance of high dose acetylsalicylic acid (ASA) in patients with concomitant active infection with varicella or influenza, or enrolling in a clinical trial; if the patient has an intermediate score, then the need for one or more interventions selected from ongoing monitoring and further testing; and if the patient has a negative score, the need for one or more interventions selected from further testing, differential diagnosis of other diseases, including measles, other viral infections (e.g., adenovirus, enterovirus), and juvenile idiopathic arthritis or other conditions including drug hypersensitivity reactions, including Stevens Johnson syndrome.

In some embodiments, the T uptake is T3 uptake or T4 uptake.

In embodiments, the method includes calculating a diagnostic score based on the concentration and weightings of protein markers determined in detection or calculation steps and classifying the diagnostic score as a positive, intermediate, or negative score; and determining Kawasaki disease in a subject as indicated by the diagnostic score. In embodiments, the method includes calculating a diagnostic score based on the concentration and weightings of protein markers determined in detection or calculation steps and classifying the diagnostic score as a positive, intermediate, or negative score; and determining MIS-C in a subject as indicated by the diagnostic score.

In embodiments, protein markers, optionally used in conjunction with clinical variables, can be used in methods for the diagnosis of Kawasaki disease. In embodiments, protein markers, optionally used in conjunction with clinical variables, can be used in methods for the diagnosis of MIS-C. In embodiments, the protein markers are selected from alpha-1 antitrypsin, alpha-1 microglobulin, angiostatin-1, apolipoprotein A, beta-2 microglobulin, brain-derived neurotrophic factor, B-type natriuretic peptide (BNP), CD163, CD5 antigen-like, clusterin, complement C3, C-reactive protein, cystatin-C, eotaxin-1, factor VII, fibrinogen, growth/differentiation factor 15, haptoglobulin, immunoglobulin A, immunoglobulin M, intercellular adhesion molecule 1, interleukin-1 alpha, interleukin-1 beta, interleukin-1 receptor antagonist, interleukin-12 subunit 40, interleukin-12 subunit 70, interleukin-17, KIM-1, MMP-3, MMP-9, Midkine, N-terminal B-type natriuretic peptide (NT-proBNP), osteocalcin, osteopontin, periostin, p-selectin, serum amyloid P-component, ST2, stem cell factor, Thyroid Hormone Uptake (T3 Uptake), Thyroid Hormone Uptake (T4 Uptake), thyroid-stimulating hormone (TSH), thyroxine (T4), thyroxine binding globulin, triiodothyronine (T3), VEGF, vitamin D-binding protein, and von Willebrand factor. In embodiments, the clinical variables are one or more of age, race, body temperature ≥38.1° C., suspicion of having Kawasaki disease, and three (3) or more days of persistent fever. For diagnosis of MIS-C, clinical variables may further include suspicion of having SARS-CoV-2 or MIS-C, and/or diagnosis of infection with SARS-CoV-2.

In embodiments, the protein markers are selected from alpha-1 antitrypsin, C Reactive Protein, interleukin-1 beta, matrix metalloproteinase-9, N-terminal prohormone of brain natriuretic peptide, periostin, thyroxine-binding globulin, and T uptake, such as T3 uptake or T4 uptake. In embodiments, the clinical value is selected from selected from one or more of age, race, body temperature ≥38.1° C., suspicion of having Kawasaki disease, and three (3) or more days of persistent fever. For diagnosis of MIS-C, clinical variables may further include suspicion of having SARS-CoV-2 or MIS-C, and/or diagnosis of infection with SARS-CoV-2.

In embodiments, the protein markers are at least two protein markers selected from alpha-1 antitrypsin, C Reactive Protein, interleukin-1 beta, matrix metalloproteinase-9, N-terminal prohormone of brain natriuretic peptide, periostin, thyroxine-binding globulin and T uptake. In embodiments, the at least two protein markers are C Reactive Protein and N-terminal prohormone of brain natriuretic peptide. In embodiments, the at least two protein markers are C Reactive Protein, N-terminal prohormone of brain natriuretic peptide and periostin. In embodiments, the at least two protein markers are alpha-1 antitrypsin, C Reactive Protein, and N-terminal prohormone of brain natriuretic peptide. In embodiments, the at least two protein markers are C Reactive Protein, matrix metalloproteinase-9, and N-terminal prohormone of brain natriuretic peptide. In embodiments, the at least two protein markers are alpha-1 antitrypsin, C Reactive Protein, matrix metalloproteinase-9, and N-terminal prohormone of brain natriuretic peptide. In embodiments, the at least two protein markers are C Reactive Protein, interleukin-1 beta, and N-terminal prohormone of brain natriuretic peptide. In embodiments, the at least two protein markers are alpha-1 antitrypsin, C Reactive Protein, interleukin-1 beta, and N-terminal prohormone of brain natriuretic peptide. In embodiments, the at least two protein markers are C Reactive Protein, N-terminal prohormone of brain natriuretic peptide and thyroxine-binding globulin. In embodiments, the at least two protein markers are alpha-1 antitrypsin, C Reactive Protein, N-terminal prohormone of brain natriuretic peptide and thyroxine-binding globulin. In embodiments, the at least two protein markers are C Reactive Protein, matrix metalloproteinase-9, N-terminal prohormone of brain natriuretic peptide and thyroxine-binding globulin. In embodiments, the at least two protein markers are C Reactive Protein, matrix metalloproteinase-9, N-terminal prohormone of brain natriuretic peptide and T uptake, as exemplified in Example 11.

In some embodiments, the at least two protein markers are selected from alpha-1 antitrypsin, C Reactive Protein, interleukin-1 beta, matrix metalloproteinase-9, N-terminal prohormone of brain natriuretic peptide, periostin, T3, T4, T3 uptake, T4 uptake, BNP, and thyroxine-binding globulin; and wherein step (i) in claim 2 comprises determining the status of at least one clinical variable selected from age, race, fever equal to or greater than 38.1° C., and persistent fever for 3 or more days.

In some embodiments, the at least two protein markers are C Reactive Protein and N-terminal prohormone of brain natriuretic peptide.

In some embodiments, at least two protein markers are C Reactive Protein, N-terminal prohormone of brain natriuretic peptide, and periostin.

In some embodiments, the at least two protein markers are alpha-1 antitrypsin, C Reactive Protein, and N-terminal prohormone of brain natriuretic peptide.

In some embodiments, the at least two protein markers are C Reactive Protein, matrix metalloproteinase-9, and N-terminal prohormone of brain natriuretic peptide.

In some embodiments, the at least two protein markers are alpha-1 antitrypsin, C Reactive Protein, matrix metalloproteinase-9, and N-terminal prohormone of brain natriuretic peptide.

In some embodiments, the at least two protein markers are C Reactive Protein, interleukin-1 beta, and N-terminal prohormone of brain natriuretic peptide.

In some embodiments, the at least two protein markers are alpha-1 antitrypsin, C Reactive Protein, interleukin-1 beta, N-terminal prohormone of brain natriuretic peptide.

In some embodiments, the at least two protein markers are C Reactive Protein, N-terminal prohormone of brain natriuretic peptide and thyroxine-binding globulin.

In some embodiments, the at least two protein markers are alpha-1 antitrypsin, C Reactive Protein, N-terminal prohormone of brain natriuretic peptide and thyroxine-binding globulin.

In some embodiments, the at least two protein markers are C Reactive Protein, matrix metalloproteinase-9, N-terminal prohormone of brain natriuretic peptide and thyroxine-binding globulin.

In some embodiments, the at least two protein markers are C Reactive Protein, N-terminal prohormone of brain natriuretic peptide and T uptake. In some embodiments, the T uptake is T3 uptake or T4 uptake.

In specific embodiments, one or more of the at least two protein markers is a protein marker previously unassociated with Kawasaki Disease. In specific embodiments, one or more of the at least two protein markers is a protein marker previously unassociated with MIS-C.

### Assay

In embodiments, the biological sample includes whole blood. In embodiments, the biological sample includes serum. In embodiments, the biological sample includes plasma.

Determining protein marker concentrations in a sample taken from a subject can be accomplished according to standard techniques known and available to the skilled artisan. In many instances, this will involve carrying out protein detection methods, which provide a quantitative measure of protein markers present in a biological sample.

In embodiments, target-binding agents that specifically bind to the protein markers described herein allow for a determination of the concentrations of the protein markers in a biological sample. Any of a variety of binding agents may be used including, for example, antibodies, polypeptides, sugars, aptamers, and nucleic acids.

In embodiments, the binding agent is an antibody or a fragment thereof that specifically binds to a protein marker as provided herein, and that is effective to determine the concentration of the protein marker to which it binds in a biological sample.

The term “specifically binds” or “binds specifically,” in the context of binding interactions between two molecules, refers to high avidity and/or high affinity binding of an antibody (or other binding agent) to a specific polypeptide subsequence or epitope of a protein marker. Antibody binding to an epitope on a specific protein marker sequence (also referred to herein as “an epitope”) is preferably stronger than binding of the same antibody to any other epitope, particularly those that may be present in molecules in association with, or in the same sample, as the specific protein marker of interest. Antibodies which bind specifically to a protein marker of interest may be capable of binding other polypeptides at a weak, yet detectable, level (e.g., 10% or less, 5% or less, 1% or less of the binding shown to the polypeptide of interest). Such weak binding, or background binding, is readily discernible from the specific antibody binding to the compound or polypeptide of interest, e.g., by use of appropriate controls. In general, antibodies used in compositions and methods described herein which bind to a specific protein marker protein with a binding affinity of 107 moles/L or more, preferably 108 moles/L or more are said to bind specifically to the specific protein marker protein.

In embodiments, the affinity of specific binding of an antibody or other binding agent to a protein marker is about 2 times greater than background binding, about 5 times greater than background binding, about 10 times greater than background binding, about 20 times greater than background binding, about 50 times greater than background binding, about 100 times greater than background binding, or about 1000 times greater than background binding, or more.

In embodiments, the affinity of specific binding of an antibody or other binding agent to a protein marker is between about 2 to about 1000 times greater than background binding, between about 2 to 500 times greater than background binding, between about 2 to about 100 times greater than background binding, between about 2 to about 50 times greater than background binding, between about 2 to about 20 times greater than background binding, between about 2 to about 10 times greater than background binding, or any intervening range of affinity.

In embodiments, the concentration of a protein marker is determined using an assay or format including, but not limited to, e.g., immunoassays, ELISA sandwich assays, lateral flow assays, flow cytometry, mass spectrometric detection, calorimetric assays, binding to a protein array (e.g., antibody array), single molecule detection methods, nanotechnology-based detection methods, or fluorescent activated cell sorting (FACS). In some embodiments, an approach involves the use of labeled affinity reagents (e.g., antibodies, small molecules, etc.) that recognize epitopes of one or more protein marker proteins in an immunoassay, an ELISA, antibody-labelled fluorescent bead array, antibody array, or FACS screen. As noted, any of a number of illustrative methods for producing, evaluating and/or using antibodies for detecting and quantifying the protein markers herein are well known and available in the art. It will also be understood that the protein detection and quantification in accordance with the methods described herein can be carried out in single assay format, multiplex format, or other known formats.

In embodiments, the concentration of a given protein is normalized to a quantification standard. In embodiments, the quantification standard is synthetic. A number of normalization methods are known in the art.

A number of suitable high-throughput multiplex formats exist for evaluating the disclosed protein markers. Typically, the term “high-throughput” refers to a format that performs a large number of assays per day, such as at least 100 assays, 1000 assays, up to as many as 10,000 assays or more per day. When enumerating assays, either the number of samples or the number of markers assayed can be considered.

In embodiments, the samples are analyzed on an assay system or analytical device. For example, the assay system or analytical device may be a multiplex analyzer that simultaneously measures multiple analytes, e.g., proteins, in a single microplate well. The assay format may be receptor-ligand assays, immunoassays, and enzymatic assays. An example of such an analyzer is the Luminex® 100/200 system which is a combination of three xMAP® Technologies. The first is xMAP microspheres, a family of fluorescently dyed micron-sized polystyrene microspheres that act as both the identifier and the solid surface to build the assay. The second is a flow cytometry-based instrument, the Luminex® 100/200 analyzer, which integrates key xMAP® detection components, such as lasers, optics, fluidics, and high-speed digital signal processors. The third component is the xPONENT® software, which is designed for protocol-based data acquisition with robust data regression analysis.

By determining protein marker levels and optionally clinical variable status for a subject, a dataset may be generated and used (as further described herein) to classify the biological sample to one or more of diagnosis, prognosis, and monitoring of the disease status of the subject, and further assigning a likelihood of a positive, intermediate, or negative diagnosis, outcome, or one or more future changes in Kawasaki disease status to the subject to thereby establish a diagnosis, prognosis, and/or monitoring of Kawasaki disease and/or outcome, as described herein. By determining protein marker levels and optionally clinical variable status for a subject, a dataset may be generated and used (as further described herein) to classify the biological sample to one or more of diagnosis, prognosis, and monitoring of the disease status of the subject, and further assigning a likelihood of a positive, intermediate, or negative diagnosis, outcome, or one or more future changes in MIS-C status to the subject to thereby establish a diagnosis, prognosis, and/or monitoring of MIS-C and/or outcome, as described herein. Of course, the dataset may be obtained via automation or manual methods.

In some embodiments, the methods provided herein may be used for the diagnosis, prognosis, and monitoring of the disease status in a subject who has been diagnosed with SARS-CoV-2 infection, COVID-19, and/or Multi-System Inflammatory Syndrome in Children (MIS-C). In some embodiments, the methods provided herein may include steps needed for diagnosing a current SARS-CoV-2 infection. Such steps may include, but are not necessarily limited to, carrying out a real-time qRT-PCR assay to detect the presence of SARS-CoV-2 RNA. In some embodiments, the step needed for diagnosing a SARS-CoV-2 infection may include, but is not necessarily limited to, carrying out a SARS-CoV-2 antigen test. In some embodiments, the step needed for diagnosing SARS-CoV-2 infection may include, but is not necessarily limited to, carrying out a plaque assay to detect the presence of SARS-CoV-2 infectious virus. In some embodiments, the methods provided herein may include steps needed for detecting a past SARS-CoV-2 infection. Such steps may include, but are not necessarily limited to, performing an immunoassay like an ELISA to detect the presence of SARS-CoV-2 antibodies. In some embodiments, the methods provided herein allow for detection of Kawasaki Disease and/or Multi-System Inflammatory Syndrome in Children (MIS-C) that may have resulted from SARS-CoV-2 infection and/or COVID-19.

### Statistical Analysis

By analyzing combinations of protein markers and optional clinical variables as described herein, the methods described herein are capable of discriminating between different endpoints. The endpoints may include, for example, incomplete or full Kawasaki disease; or MIS-C. The identity of the markers and their corresponding features (e.g., concentration, quantitative levels) are used in developing and implementing an analytical process, or plurality of analytical processes, that discriminate between clinically relevant classes of patients.

Methods described herein may utilize machine learning. Machine learning is a field of statistics and computer science where algorithms generate models from data for the sake of prediction, regression, or classification. Machine learning algorithms generally require a set of “features”, which are the variables that are used to predict an “outcome” or “class”. In our case, the features are the transformed, normalized protein levels or concentrations and, optionally, the clinical factors, and the class or outcome is the medical outcome that is to be predicted and treated. The accuracy of learning models can be evaluated with many different metrics, depending on the type of class that the model is trying to predict different metrics will be used for a binary outcome (e.g., “positive” vs. “negative”), tertiary outcome (“positive”, “intermediate”, or “negative”) or a continuous numeric outcome. Machine learning gives computers the ability to learn without being explicitly programmed. Machine learning explores the study and construction of algorithms that can learn from and make predictions on data—such algorithms overcome following strictly static program instructions by making data-driven predictions or decisions through building a model from sample inputs. Machine learning is employed in a range of computing tasks where designing and programming explicit algorithms with good performance is difficult or infeasible. As a scientific endeavor, machine learning grew out of the quest for artificial intelligence (AI) and is considered a subset of artificial intelligence. Already in the early days of AI, some researchers were interested in having machines learn from data. They attempted to approach the problem with various symbolic methods, as well as what were then termed “neural networks”. Probabilistic reasoning was also employed, especially in automated medical diagnosis.

A protein marker and, optionally, clinical variable dataset may be used in an analytic process for correlating the assay result(s) generated by the assay system and optionally the clinical variable status to the disease status of the subject, wherein said correlation step comprises correlating the assay result(s) to one or more of risk stratification, diagnosis, prognosis, classifying and monitoring of Kawasaki disease in the subject, where the correlating step includes assigning a likelihood of a positive, intermediate, or negative diagnosis, or one or more future changes in disease status to the subject based on the assay result(s).

A protein marker and, optionally, clinical variable dataset may be used in an analytic process for correlating the assay result(s) generated by the assay system and optionally the clinical variable status to the disease status of the subject, wherein said correlation step comprises correlating the assay result(s) to one or more of risk stratification, diagnosis, prognosis, classifying and monitoring of MIS-C in the subject, where the correlating step includes assigning a likelihood of a positive, intermediate, or negative diagnosis, or one or more future changes in disease status to the subject based on the assay result(s).

A protein marker and, optionally, clinical variable dataset may be used in an analytic process for generating a diagnostic or prognostic result or score. For example, an illustrative analytic process can comprise a linear model with one term for each component (protein level or clinical factor) and a different weighting for each component. The result of the algorithmic model is a number that generates a diagnosis. The result may also provide a multi-level or continuous score with a higher number representing a higher likelihood of disease or risk of event, a lower number representing a lower likelihood of disease or risk of event.

The examples below illustrate how data analysis algorithms can be used to construct a number of such analytical processes. Each of the data analysis algorithms described in the examples uses features (e.g., quantitative protein levels and/or clinical factors) of a subset of the markers identified herein and different weightings for each feature across a training population. Specific data-analysis algorithms for building an analytical process or plurality of analytical processes, that discriminate between subjects disclosed herein will be described in the subsections below. Once an analytical process has been built using these example data analysis algorithms or other techniques known in the art, the analytical process can be used to classify a test subject into one of the two or more phenotypic classes after the blood test is obtained. This is accomplished by applying one or more analytical processes to one or more marker profile(s) obtained from the test subject. Such analytical processes, therefore, have enormous value as diagnostic or prognostic indicators.

In embodiments, the methods provide for an algorithm that may be used to transform the algorithmic weightings of the different concentrations of a panel of protein markers, as described above, into a score that may be used to determine whether a patient is diagnosed with incomplete or full Kawasaki disease. In embodiments, the methods provide for an algorithm that may be used to transform the algorithmic weightings of the different concentrations of a panel of protein markers, as described above, into a score that may be used to determine whether a patient is diagnosed with MIS-C.

The data are processed prior to the analytical process. The data in each dataset are collected by measuring the values for each marker, usually in duplicate or triplicate or in multiple replicates. The data may be manipulated; for example, raw data may be transformed using standard curves, and the average of replicate measurements used to calculate the average and standard deviation for each patient. These values may be transformed before being used in the models, e.g., log transformed, normalized to a standard scale, Winsorized, etc. The data is transformed via computer software. This data can then be input into the analytical process with defined parameters.

The direct concentrations of the proteins (after log transformation and normalization), the presence/absence of clinical factors represented in binary form (e.g., race), and/or clinical factors in quantitative form (e.g., body temperature, age) provide values that are entered into the algorithmically-weighted diagnostic model provided by the software, and the result is evaluated against one or more cutoffs to determine the diagnosis or prognosis of Kawasaki disease. The direct concentrations of the proteins (after log transformation and normalization), the presence/absence of clinical factors represented in binary form (e.g., race), and/or clinical factors in quantitative form (e.g., body temperature, age) provide values that are entered into the algorithmically-weighted diagnostic model provided by the software, and the result is evaluated against one or more cutoffs to determine the diagnosis or prognosis of MIS-C.

The following are examples of the types of statistical analysis methods that are available to one of skill in the art to aid in the practice of the disclosed methods, panels, assays, and kits. The statistical analysis may be applied for one or both of two tasks. First, these and other statistical methods may be used to identify preferred subsets of markers and other indices that will form a preferred dataset. In addition, these and other statistical methods may be used to generate the analytical process that will be used with the dataset to generate the result. Several statistical methods presented herein or otherwise available in the art will perform these tasks and yield a model that is suitable for use as an analytical process for the practice of the methods disclosed herein.

Prior to analysis, the data is partitioned into a training set and a validation set, if there are sufficient number of samples. The training set is used to train, evaluate and build the final diagnostic model. The validation set is not used at all during the training process and is only used to validate final diagnostic models.

The creation of training and validation sets can be done through random selection, or through chronological selection (i.e., where the training set is the first sequential set of patients, and the validation set is the second/final sequential set of patients). After these sets are determined, the balance of various outcomes is considered to confirm that the outcomes of interest are properly represented in each data set.

In cases where sample sizes are small, the entire population of patients is used to train, evaluate, and develop a diagnostic or prognostic panel. All processes below, except when explicitly mentioned, involve the use of the entire population.

The features (e.g., proteins and/or clinical factors) of the diagnostic models are selected for each outcome using a combination of analytic processes, including least angle regression (LARS; a procedure based on stepwise forward selection), shrinkage in statistical learning methods such as least absolute shrinkage and selection operator (LASSO), significance testing, and expert opinion.

The statistical learning method used to generate a result (classification, diagnosis, and/or disease/outcome risk, etc.) may be any type of process capable of providing a result useful for classifying a sample (e.g., a linear model, a probabilistic model, a decision tree algorithm, or a comparison of the obtained dataset with a reference dataset).

The diagnostic signal in the features is evaluated with these statistical learning methods using a cross-validation procedure. For each cross-validation fold, the data (either the training set or all patients, depending on the sample size) is further split into training and validation sets (hereby called CV-training and CV-validation data sets).

For each fold of cross validation, the diagnostic model is built using the CV-training data, and evaluated with the CV-validation data.

Models during the cross-validation process are evaluated with standard metrics of classification accuracy, e.g., the area under the ROC curve (AUC), sensitivity (Sn), specificity (Sp), positive predictive values (PPV), and negative predictive values (NPV).

Once a set of features (e.g., quantitative protein levels and optionally clinical factors) are selected to compose a final diagnostic or prognostic panel, a final predictive model is built using all of the training data.

Applying the patient data (e.g., transformed and normalized quantitative protein levels and/or clinical factors) into the final predictive model yields a classification result. These results can be compared against a threshold for classifying a sample within a certain class (e.g., positive, intermediate or negative diagnosis, or a severity/likelihood score).

For small populations, a final model is created with the entire population, and then this model is evaluated again with the population to determine the in-sample diagnostic (or prognostic) results.

For populations of sufficient size to warrant separation into training and validation sets, final models are evaluated with the validation data set. To respect the authority of the validation data set, it is not used in an iterative way, to feed information back into the training process. It is only used as the full stop of the analytic pipeline.

Models are evaluated with the entire population (for smaller populations) or with the validation data set (for populations of sufficient size to warrant separation into training and validation set), using metrics of diagnostic (or prognostic) accuracy, including the area-under-the-curve (AUC), sensitivity, specificity, positive predictive value and/or negative predictive value. Other metrics of accuracy, such as hazard ratio, relative risk, and net reclassification index are considered separately for models of interest.

This final model or a model optimized for a particular protein marker platform, when used in a clinical setting, may be implemented as a software system, running directly on the assay hardware platform or on an independent system. The model may receive protein level or concentration data directly from the assay platform or other means of data transfer, and patient clinical data may be received via electronic, manual, or other query of patient medical records or through interactive input with the operator. This patient data may be processed and run through the final model, which will provide a result to clinicians and medical staff for purposes of decision support.

In embodiments, the protein markers and/or clinical variables include those listed in Table 1, particularly those that are associated with a p-value of less than 0.1, less than 0.05, less than 0.01 or less than 0.001.

In some embodiments, at least 2, at least 3 or at least 4 protein markers are used in the methods provided herein. In other embodiments, the number of protein markers employed can vary, and may include at least 5, 6, 7, 8, 9, 10, or more. In still other embodiments, the number of protein markers can include at least 15, 20, 25 or 50, or more.

In embodiments, the methods provided herein include measuring the concentrations of at least two protein markers selected from Table 1. Such determination can be made by standard methods known in the art and described herein. In embodiments, measurement of the concentrations of at least two protein markers selected from Table 1 determines a subject's protein profile.

In embodiments, the analytical device for measuring the concentrations of protein markers is an immunoassay device. The device may be configured with software controls and analytical programs capable of mathematical computations such as normalizing detected protein marker concentrations against a quantification standard. The quantification standard may be part of the protein detection assay or may be separately contained. The software controls and analytical programs may be further capable of transform the normalized concentrations into a score based on pre-entered algorithms and models to accept the protein marker concentrations and the optional clinical variable(s).

In embodiments, the status of at least one clinical variable selected from Table 2 is determined. Such determination can be made by standard methods known in the art such as medical history review or from the analytical device retrieving the clinical variable(s) from other means, including but not limited to electronic health records (EHR) or other information systems.

In embodiments, assigning a score to the subject based on the protein marker profile and optionally the clinical value status can be accomplished using a device configured with software controls and analytical programs capable of mathematical computations as described above. The score may be classified as a positive or negative diagnostic result. The score may be classified as a positive, intermediate, or negative diagnostic result. The score may be classified as a positive or negative prognostic result. The score may be classified as a positive, intermediate, or negative prognostic result.

### Scoring and Treatments

In some embodiments, the diagnostic calculations will result in a numeric or categorical score that relates the patient's diagnosis of incomplete or full Kawasaki disease. The number of levels used by the diagnostic model may be as few as two (“positive” vs. “negative”) or as many as deemed clinically relevant, where a higher score indicates a higher likelihood of disease. Specifically, a score of 1 indicates a strong degree of confidence in a low likelihood of Kawasaki disease or a negative result, a score of 5 indicates a strong degree of confidence in a high likelihood of Kawasaki disease or a positive result (determined by the test's PPV or Sp), and a score of 3 indicates an intermediate or incomplete Kawasaki disease.

In some embodiments, the diagnostic calculations will result in a numeric or categorical score that relates the patient's diagnosis of MIS-C. The number of levels used by the diagnostic model may be as few as two (“positive” vs. “negative”) or as many as deemed clinically relevant, where a higher score indicates a higher likelihood of disease. Specifically, a score of 1 indicates a strong degree of confidence in a low likelihood of MIS-C or a negative result, a score of 5 indicates a strong degree of confidence in a high likelihood of MIS-C or a positive result (determined by the test's PPV or Sp), and a score of 3 indicates an intermediate or incomplete MIS-C.

In embodiments, a positive diagnostic score in the subject facilitates a determination by a medical practitioner of the need for one or more interventions selected from administration of pharmacological agents, echocardiography, and avoidance of high dose acetylsalicylic Acid (ASA) in patients with concomitant active infection with varicella or influenza. In embodiments, the pharmacological agent is one or more of intravenous immunoglobulin, acetylsalicylic Acid (ASA), methylprednisolone, and infliximab.

In embodiments, an intermediate diagnostic score in the subject facilitates a determination by a medical practitioner of the need for one or more interventions selected from further testing and on-going monitoring of the subject.

In embodiments, a negative diagnostic score in the subject facilitates a determination by a medical practitioner of the need for one or more interventions selected from further testing, differential diagnosis of other diseases, including measles, other viral infections (e.g., adenovirus, enterovirus), and juvenile idiopathic arthritis or other conditions including drug hypersensitivity reactions, including Stevens Johnson syndrome.

Compositions for treatment may be administered to the subject in a number of ways depending on whether local or systemic treatment is desired. Thus for example, the treatment may be administered intravenously. In embodiments, the treatment may be administered via an intra-articular injection. In embodiments, the treatment may be administered via inhalation. In embodiments, the treatment may be administered via nebulization. In embodiments, the treatment may be administered intranasally, orally, by inhalation, vaginally, rectally, or parenterally, for example by intradermal, subcutaneous, intramuscular, intraperitoneal, intrarectal, intraarterial, intralymphatic, intravenous, intra-articular, intrathecal, and intratracheal routes. Parenteral administration, if used, is generally characterized by injection.

In some embodiments, the present disclosure provides a method of administering a therapeutic intervention to a subject suspected of having Kawasaki disease comprising determining the subject's protein marker profile for a panel of protein markers comprising at least two protein markers selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor, and, optionally, determining the status of at least one clinical variable for the subject, wherein the clinical variable is age, race, fever ≥38.1° C., suspicion of Kawasaki disease, or persistent fever for 3 or more days; assigning a score to the subject based on the protein marker profile in (i) and the clinical value status in (ii) wherein the score is classified as a positive, intermediate, or negative score, said score algorithmically-derived from normalized and mathematically transformed concentrations of protein markers in the subject's sample and optionally, the status of at least one clinical variable; and administering to the subject a therapeutic intervention based on the positive, intermediate or negative score.

In some embodiments, the present disclosure provides a method of administering a therapeutic intervention to a subject suspected of having MIS-C comprising determining the subject's protein marker profile for a panel of protein markers comprising at least two protein markers selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor, and optionally, determining the status of at least one clinical variable for the subject, wherein the clinical variable is age, race, fever ≥38.1° C., suspicion of MIS-C, suspicion of SARS-CoV-2 infection, diagnosis of SARS-CoV-2 infection, or persistent fever for 3 or more days; assigning a score to the subject based on the protein marker profile in (i) and the clinical value status in (ii) wherein the score is classified as a positive, intermediate, or negative score, said score algorithmically-derived from normalized and mathematically transformed concentrations of protein markers in the subject's sample and optionally, the status of at least one clinical variable; and administering to the subject a therapeutic intervention based on the positive, intermediate or negative score.

### Panels, Assays, and Kits

The present disclosure further provides panels, assays, and kits including target-binding agents that bind at least two or greater than two protein markers, a synthetic standard, and optionally clinical variable(s) set forth in Table 2, in order to aid or facilitate a diagnostic finding according to the present disclosure. For example, in embodiments, a diagnostic panel or kit includes a plurality of protein markers set out in Table 1, a synthetic standard, and optionally one or a plurality of applicable clinical variables set out in Table 2. In embodiments, the panel includes target-binding agents for one or more of alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor.

It will be understood that, in embodiments, the panels, assays, and kits described herein include antibodies, binding fragments thereof and/or other types of binding agents which are specific for one or more of the protein markers of Table 1 and which are useful for determining the concentrations of the corresponding protein marker in a biological sample according to the methods describe herein. Accordingly, in each description herein of a panel, assay, or kit comprising one or a plurality of protein markers, it will be understood that the very same panel, assay, or kit can advantageously include, in addition or instead, one or a plurality of antibodies, binding fragments thereof or other types of target binding agents such as aptamers, which are specific for a protein marker as set forth in Table 1. Of course, the panels, assays, and kits can further comprise, include or recommend a determination of one or a plurality of applicable clinical variables as set out in Table 2.

In embodiments, the protein markers and/or clinical variables used in conjunction with a panel, assay, or kit include those listed in Table 1, and Table 2 respectively, particularly those which are associated with a p-value of less than 0.1, less than 0.05, less than 0.01 or less than 0.001.

In embodiments, panels, assays, and kits may include at least two target-binding agents specific for protein markers as described herein. In embodiments, panels, assays, and kits may include target-binding agents for two protein markers. In embodiments, panels, assays, and kits may comprise target-binding agents for three protein markers. In embodiments, panels, assays, and kits may comprise target-binding agents for four protein markers. In embodiments, panels, assays, and kits may comprise target-binding agents for five protein markers. In other embodiments, the number of protein markers employed can include at least 6, 7, 8, 9 or 10 or more. In still other embodiments, the number of protein markers employed can include at least 15, 20, 25, 30 or 35, or more.

As described herein, panels, assays, and kits of the present disclosure can be used for diagnosing incomplete or full Kawasaki disease. As described herein, panels, assays, and kits of the present disclosure can be used for diagnosing MIS-C

In embodiments, a panel, assay, or kit is used to in the evaluation of a subject's positive, intermediate, or negative response to a therapeutic and/or intervention for Kawasaki disease. In specific embodiments, a panel, assay, or kit for the diagnosis of Kawasaki disease includes target-binding agents for at least two protein markers selected from alpha-1 antitrypsin, C Reactive Protein, interleukin-1 beta, matrix metalloproteinase-9, N-terminal prohormone of brain natriuretic peptide, periostin thyroxine-binding globulin, and T uptake a synthetic standard, and optionally, one or a plurality of applicable clinical variables set out in Table 2.

In embodiments, a panel, assay, or kit is used in the evaluation of a subject's positive, intermediate, or negative response to a therapeutic and/or intervention for MIS-C. In specific embodiments, a panel, assay, or kit for the diagnosis of MIS-C includes target-binding agents for at least two protein markers selected from alpha-1 antitrypsin, C Reactive Protein, interleukin-1 beta, matrix metalloproteinase-9, N-terminal prohormone of brain natriuretic peptide, periostin thyroxine-binding globulin, and T uptake a synthetic standard, and optionally, one or a plurality of applicable clinical variables set out in Table 2, and/or suspicion of having MIS-C and/or SARS-CoV-2.

In embodiments, a panel, assay, or kit comprises at least 2, at least 3, at least 4 or greater than 4 antibodies or binding fragments thereof, or other types of binding agents, where the antibodies, binding fragments or other binding agents are specific for a protein marker of Table 1.

It will be understood that the panels, assays, and kits of the present disclosure may further comprise virtually any other compounds, compositions, components, instructions, or the like, that may be necessary or desired in facilitating a determination of a diagnosis according to the present disclosure. These may include instructions for using the panel, assay, or kit, instructions for making a diagnostic or prognostic determination (e.g., by calculating a diagnostic score), instructions or other recommendations for a medical practitioner in relation to preferred or desired modes of therapeutic or diagnostic intervention in the subject in light of the diagnostic and/or monitoring therapeutic effects and the like.

In some embodiments, the panels, assays, and kits as described herein will facilitate detection of the protein markers discussed herein. Means for measuring such blood, plasma and/or serum concentrations are known in the art, and include, for example, the use of an immunoassay.

In addition to the methods described above, any method known in the art for quantitatively measuring levels of protein in a sample, e.g., non-antibody-based methods can be used in the methods and kits as described herein. For example, mass spectrometry-based (such as, for example, Multiple Reaction Monitoring (MRM) mass spectrometry) or HPLC-based methods can be used.

In some embodiments, the panels, assays, and kits provided herein may include reagents for diagnosing SARS-CoV-2 infection. Such reagents may include, but are not necessarily limited to, primers, probes, buffer solutions, polymerases, nucleotides, control templates, and PCR plates needed to perform a diagnostic PCR assay to detect the presence of viral RNA. In some embodiments, reagents may include a SARS-CoV-2 antigen test and related buffers. In some embodiments, reagents may include components necessary for performing an assay aimed at detecting infectious virus, such as including, but not necessarily limited to, a plaque assay. In some embodiments, reagents may include components needed to screen for antibodies against SARS-CoV-2. Such reagents may include, but are not necessarily limited to, reagents needed to perform an immunoassay like an ELISA. In some embodiments, the panels, assays, and kits provided herein allow for detection of current or past SARS-CoV-2 infection or COVID-19. In some embodiments, the panels, assays, and kits provided herein allow for detection of Kawasaki Disease and/or Multi-System Inflammatory Syndrome in Children (MIS-C) that may have resulted from SARS-CoV-2 infection and/or COVID-19.

Additionally, technologies such as those used in the field of proteomics and other areas may also be embodied in methods, kits and other aspects as described herein. Such technologies include, for example, the use of micro- and nano-fluidic chips, biosensors and other technologies as described, for example, in United States Patent Application Nos. US2008/0202927; US2014/0256573; US2016/0153980; WO2016/001795; US2008/0185295; US2010/0047901; US2010/0231242; US2011/0154648; US2013/0306491; 052010/0329929; US2013/0261009; each of which is incorporated herein by reference in its entirety.

### EXAMPLES

### Example 1: A Protein Marker Scoring System to Diagnose Kawasaki Disease (KD), Panel KDA 001

Patients were selected on the basis of established criteria for either complete or incomplete Kawasaki Disease.

After informed consent was obtained, detailed clinical and historical variables were recorded.

Two (2) to three (3) mL of blood were obtained upon enrollment. The blood was immediately centrifuged for 15 minutes, serum and plasma aliquoted on ice and frozen at −80° C. until biomarker measurement.

After a single freeze-thaw cycle, approximately 200 μl of plasma or serum was analyzed for 42 of the 48 protein biomarkers on a Luminex 100/200 xMAP technology platform. This technology utilizes multiplexed, microsphere-based assays in a single reaction vessel. It combines optical classification schemes, biochemical assays, flow cytometry and advanced digital signal processing hardware and software. Multiplexing is accomplished by assigning each protein-specific assay a microsphere set labeled with a unique fluorescence signature. An assay-specific capture antibody is conjugated covalently to each unique set of microspheres. The assay-specific capture antibody on each microsphere binds the protein of interest. A cocktail of assay-specific, biotinylated detecting antibodies is reacted with the microsphere mixture, followed by a streptavidin-labeled fluorescent “reporter” molecule. Similar to a flow cytometer, as each individual microsphere passes through a series of excitation beams, it is analyzed for size, encoded fluorescence signature and the amount of fluorescence generated is proportionate to the protein level. A minimum of 100 individual microspheres from each unique set are analyzed and the median value of the protein-specific fluorescence is logged. Using internal controls of known quantity, sensitive and quantitative results are achieved with precision enhanced by the analysis of 100 microspheres per data point. Separately, plasma or serum was analyzed for 5 proteins on a Siemens Dimension Vista. This platform utilizes luminescent oxygen channeling (LOCI) and is a homogeneous immunoassay method. Latex particle pairs are formed in the assay through specific binding interactions by sequentially combining the sample and two reagents. One particle contains a photosensitizer, the other contains a chemiluminescer. Irradiation causes photosensitized formation of singlet oxygen, which migrates to a bound particle and activates the chemiluminescer, thereby initiating a delayed luminescence emission. Assay times range from one to 25 minutes.

The patients selected for analysis consisted of the 50 patients with a Kawasaki disease diagnosis (“cases”) and the 100 febrile patients who were admitted but eventually ruled out for Kawasaki disease (“controls”).

Because of the relatively small number of patients available, all of them were selected to be used for analysis (i.e., they were not partitioned into a training and a validation set). Baseline clinical characteristics and protein concentrations between cases and controls were compared; dichotomous variables were compared using two-sided Fisher's exact test, while continuous variables were compared using two-sided two-sample T test. The biomarkers compared were tested with the Wilcoxon Rank Sum test, as their concentrations were not normally distributed. For any marker result that was unmeasurable, a standard approach of imputing concentrations 50% below the limit of detection was utilized.

All work for biomarker selection and the development of a diagnostic model was done on all of the 150 patients selected for the analysis. The level or concentration values for all proteins underwent the following transformation to facilitate the predictive analysis: (a) they were log transformed to achieve a normal distribution; (b) outliers were clipped at the value of three times the median absolute deviation; and (c) the values were re-scaled to distribution with a zero mean and unit variance. Machine learning statistical techniques, a subset of artificial intelligence, were utilized. Candidate panels of proteins from Table 1 and optionally clinical features from Table 2 were selected via least angle regression (LARS), and models were generated using least absolute shrinkage and selection operator (LASSO) with logistic regression, using Monte Carlo cross-validation with 400 iterations. Candidates were subjected to further assessment of discrimination via iterative model building, assessing change in area under the curve (AUC) with the addition of biomarkers to the base model, along with assessment of improvement in calibration from their addition through minimization of the Akaike or Bayesian Information Criteria (AIC, BIC) and goodness of fit in Hosmer-Lemeshow testing.

Once the final panel was selected, a final model was built with the data from the entire population. Multivariable logistic regression evaluated the performance of the model in the population as a whole as well as in several relevant subgroups, to determine how well the model performed in men vs. women and correcting for age. A score distribution was generated within the population, followed by receiver operator characteristic (ROC) testing with valor of the score as a function of the AUC. Operating characteristics of the score were calculated, with sensitivity (Sn), specificity (Sp), positive and negative predictive value (PPV, NPV) generated.

All statistics were performed using R software, version 3.3 or later (R Foundation for Statistical Computing, Vienna, AT); p-values are two-sided, with a value <0.05 considered significant.

Following the described methods, independent predictors of Kawasaki disease included seven biomarkers (alpha-1 antitrypsin, C Reactive Protein, interleukin-1 beta, matrix metalloproteinase-9, N-terminal prohormone of brain natriuretic peptide, periostin, thyroxine-binding globulin and T uptake). In this example panel, C Reactive Protein and N-terminal prohormone of brain natriuretic peptide were analyzed (Table 3, analysis KDA 001).

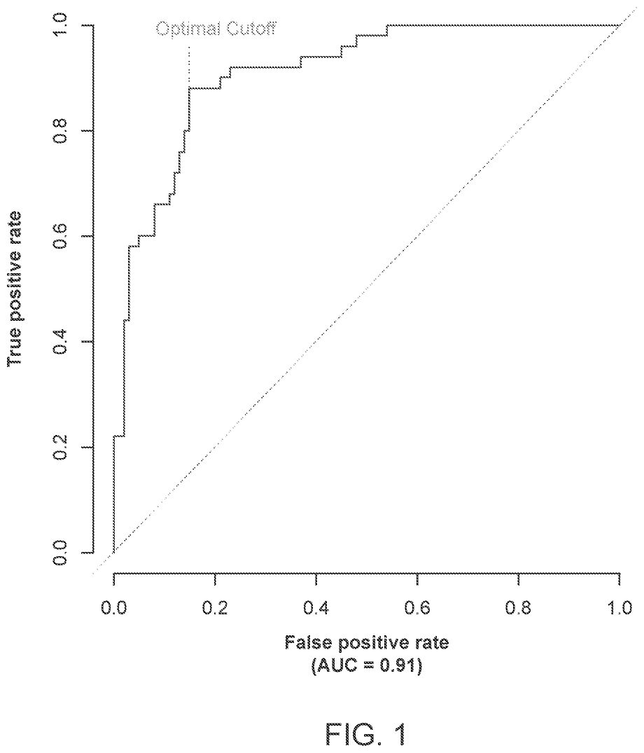

Individual scores were calculated and results were expressed as a function of Kawasaki disease presence. In doing so, a bimodal score distribution was revealed, with higher prevalence of Kawasaki disease in those with higher scores, and lower prevalence among those with lower scores. In ROC testing the scores generated had a cross-validated AUC of 0.91, and an in-sample AUC of 0.91 (Table 3; FIG. 1; p<0.001).

The biomarker-based scoring strategy disclosed herein can reliably diagnose the presence of Kawasaki disease. Advantages of a reliable biomarker, and optionally clinical, score for diagnosing Kawasaki disease include the fact such a technology can be widely disseminated in a cost-effective manner, easily interpreted, and are associated with a well-defined sequence of therapeutic steps.

### Example 2: A Protein Marker Scoring System to Diagnose Kawasaki Disease (KD), Panel KDA 030

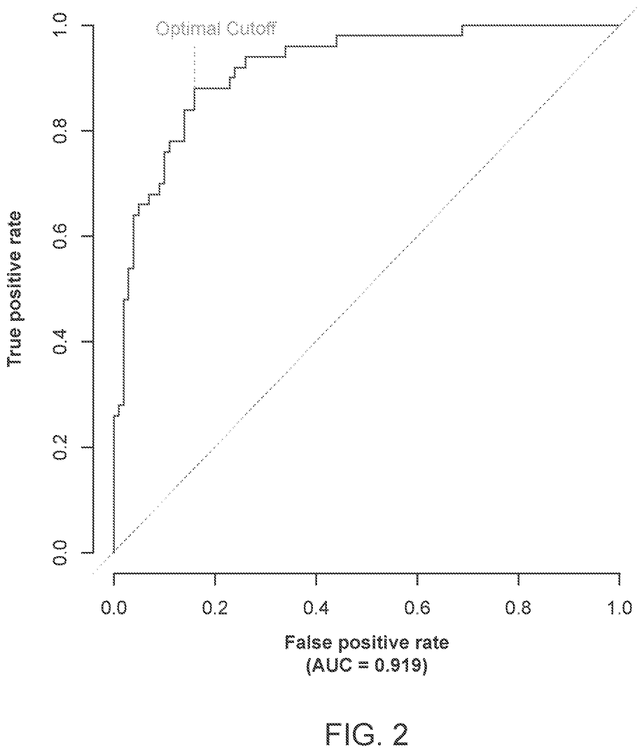

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 2)) as Example 1.

### Example 3: A Protein Marker Scoring System to Diagnose Kawasaki Disease (KD), Panel KDA 080

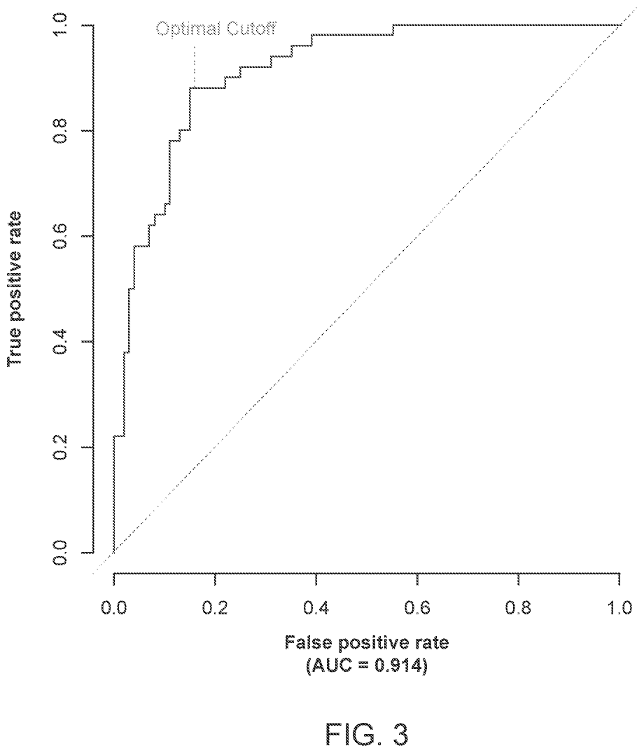

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 3)) as Example 1.

### Example 4: A Protein Marker Scoring System to Diagnose Kawasaki Disease (KD), Panel KDA 038

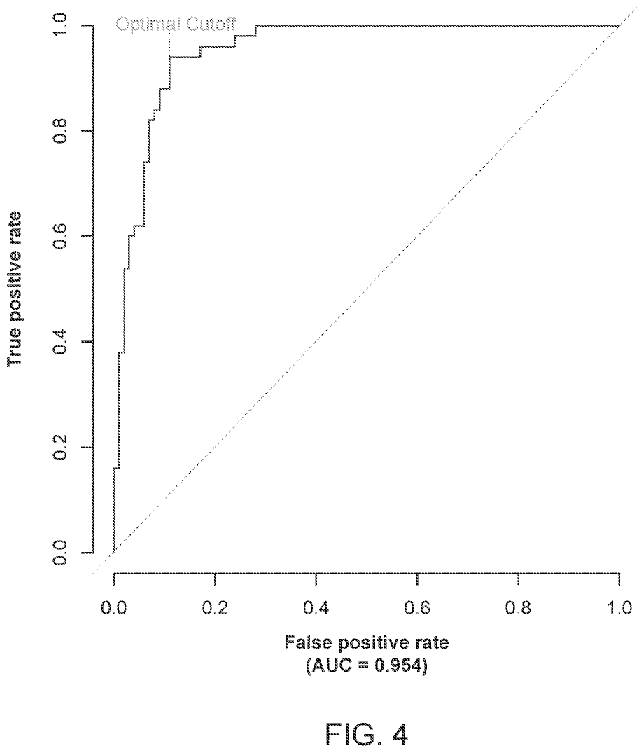

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 4)) as Example 1.

### Example 5: A Protein Marker Scoring System to Diagnose Kawasaki Disease (KD), Panel KDA 089

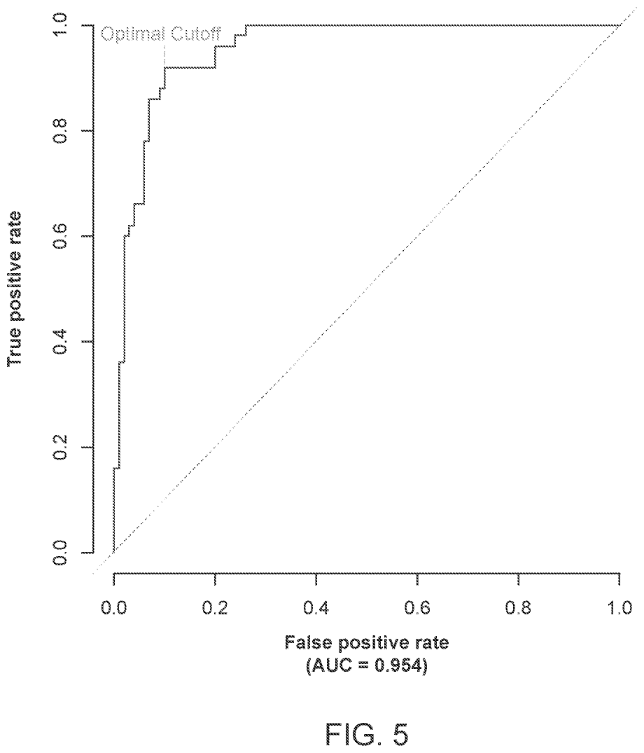

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 5)) as Example 1.

### Example 6: A Protein Marker Scoring System to Diagnose Kawasaki Disease (KD), Panel KDA 017

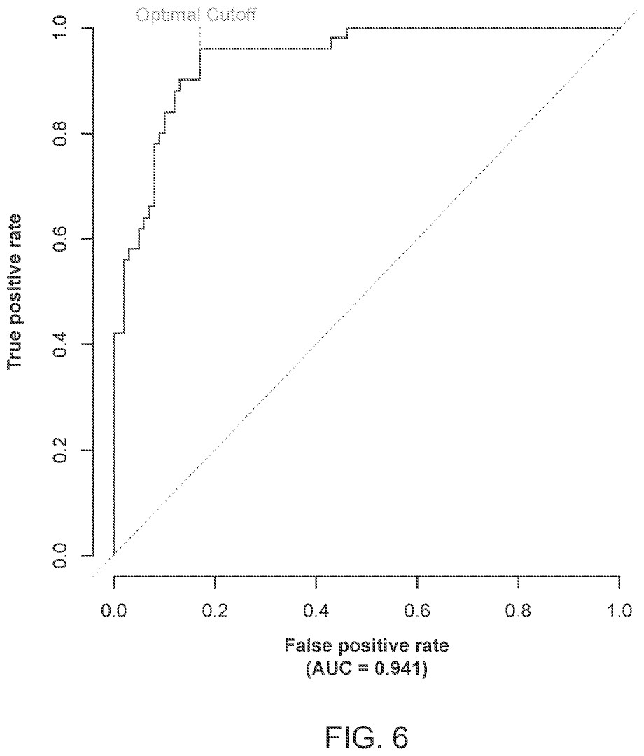

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 6)) as Example 1.

### Example 7: A Protein Marker Scoring System to Diagnose Kawasaki Disease (KD), Panel KDA 086

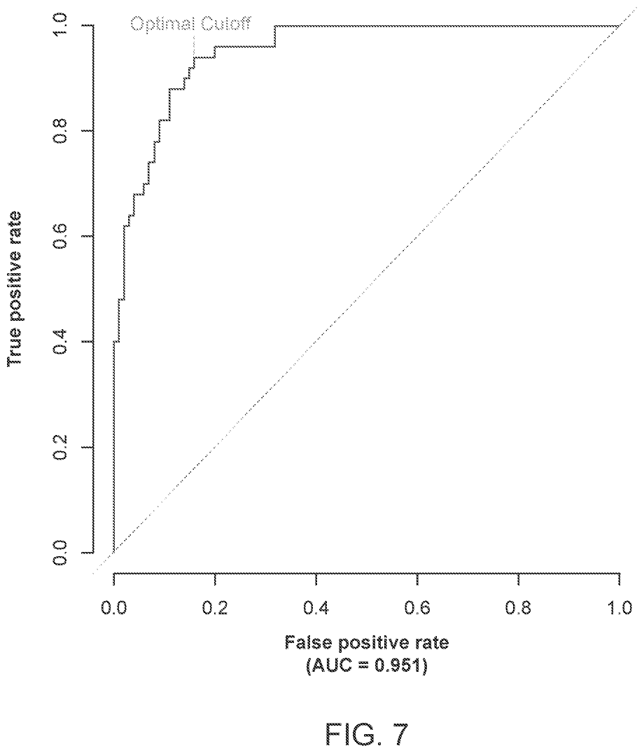

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 7)) as Example 1.

### Example 8: A Protein Marker Scoring System to Diagnose Kawasaki Disease (KD), Panel KDA 012

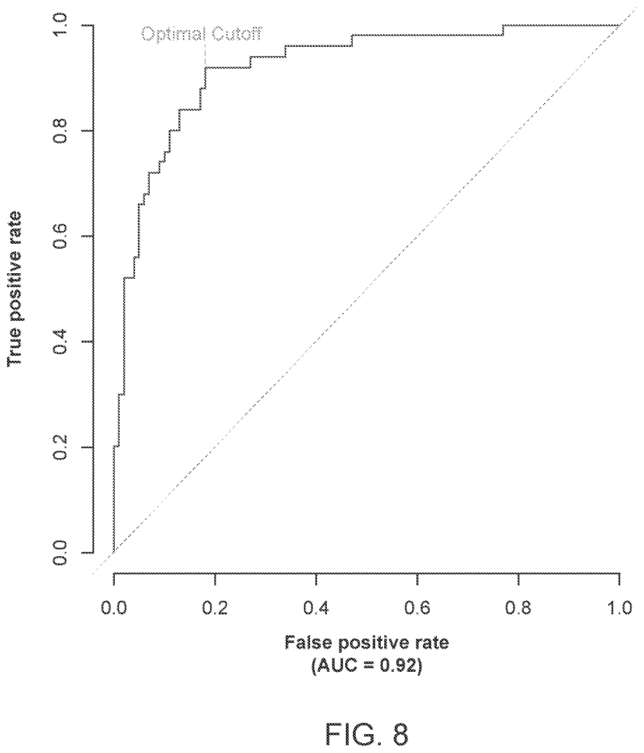

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 8)) as Example 1.

### Example 9: A Protein Marker Scoring System to Diagnose Kawasaki Disease (KD), Panel KDA 085

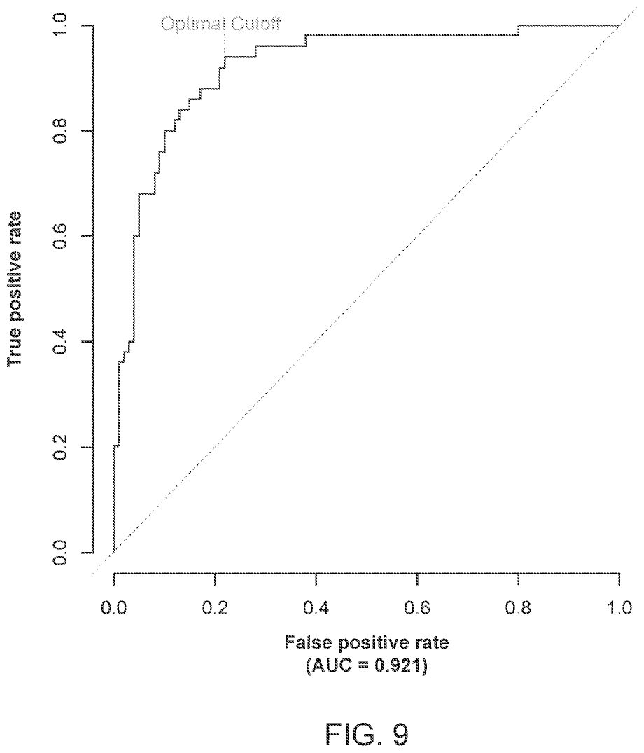

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 9)) as Example 1.

### Example 10: A Protein Marker Scoring System to Diagnose Kawasaki Disease (KD), Panel KDA 043

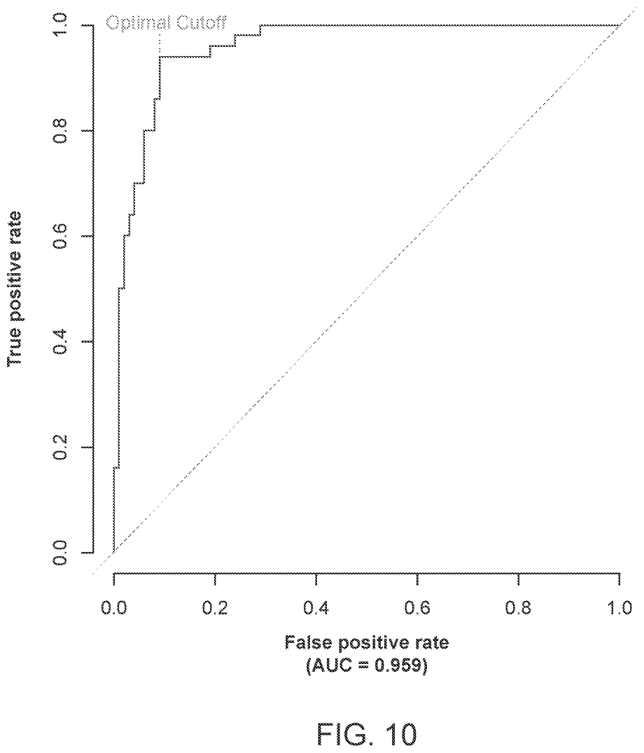

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 10)) as Example 1.

### Example 11: A Protein Marker Scoring System to Diagnose Kawasaki Disease (KD), Panel KDA U007

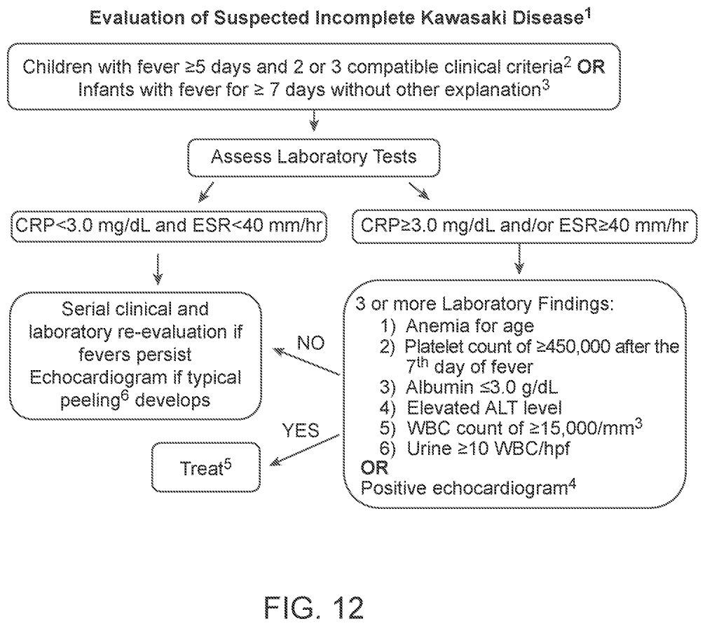

This example demonstrates yet another non-invasive method employing a protein marker scoring system that offers, among other things, high accuracy in providing a diagnosis of Kawasaki disease. This example utilized the same described methods (study design and participants, data acquisition, follow up, protein marker testing, statistics and results (Tables 1, 2, and 3 and FIG. 12)) as Example 1.

### Example 12: Further Demonstration of Methods Employing Clinical and Protein Marker Analysis for the Diagnosis of Kawasaki Disease

Table 3 is a chart of the different panels comprising protein markers and optionally clinical variables with corresponding AUCs for the given outcome. These reflect aforementioned Examples 1-11.

TABLE 3
Performance of Different Panels for Various Outcomes Comprising Protein Markers
and Optionally Clinical Variables with Corresponding AUCs and Figures

| | Test | | | | |
|---|---|---|---|---|---|
| | Outcome/ | | Cross | In | |
| Analysis | Positive | Protein markers & | Validated | Sample/Entire | Figure |
| # | Endpoint | Clinical Variables | Mean AUCs | Population | Reference |
| KDA 001 | Diagnostic | C Reactive Protein, | 0.91 | 0.91 (rounded | 1 |
| Example 1 | Panels for | N-terminal prohormone | | from 0.9102) | |
| | Kawasaki | of brain natriuretic | | | |
| | Disease | peptide | | | |
| KDA 030 | Diagnosis for | C Reactive Protein, | 0.91 | 0.92 (rounded | 2 |
| Example 2 | Kawasaki | N-terminal prohormone | | from 0.9188) | |
| | Disease | of brain natriuretic | | | |
| | | peptide, periostin | | | |
| KDA 080 | Diagnosis for | alpha-1 antitrypsin, | 0.90 | 0.91 (rounded | 3 |
| Example 3 | Kawasaki | C Reactive Protein, | | from 0.9144) | |
| | Disease | N-terminal prohormone | | | |
| | | of brain natriuretic | | | |
| | | peptide | | | |
| KDA 038 | Diagnosis for | C Reactive Protein, matrix | 0.95 | 0.95 (rounded | 4 |
| Example 4 | Kawasaki | metalloproteinase-9, N- | | from 0.9536) | |
| | Disease | terminal prohormone of | | | |
| | | brain natriuretic peptide | | | |
| KDA 089 | Diagnosis for | alpha-1 antitrypsin, C | 0.95 | 0.95 (rounded | 5 |
| Example 5 | Kawasaki | Reactive Protein, matrix | | from 0.9544) | |
| | Disease | metalloproteinase-9, | | | |
| | | N-terminal prohormone of | | | |
| | | brain natriuretic peptide | | | |
| KDA 017 | Diagnosis for | C Reactive Protein, | 0.94 | 0.94 (rounded | 6 |
| Example 6 | Kawasaki | interleukin-1 beta, | | from 0.9412) | |
| | Disease | N-terminal prohormone | | | |
| | | of brain natriuretic | | | |
| | | peptide | | | |
| KDA 086 | Diagnosis for | alpha-1 antitrypsin, | 0.94 | 0.95 (rounded | 7 |
| Example 7 | Kawasaki | C Reactive Protein, | | from 0.9510) | |
| | Disease | interleukin-1 beta | | | |
| | | N-terminal prohormone | | | |
| | | of brain natriuretic | | | |
| | | peptide | | | |
| KDA 012 | Diagnosis for | C Reactive Protein, | 0.91 | 0.92 (rounded | 8 |
| Example 8 | Kawasaki | N-terminal prohormone | | from 0.9196) | |
| | Disease | of brain natriuretic | | | |
| | | peptide, thyroxine- | | | |
| | | binding globulin | | | |
| KDA 085 | Diagnosis for | alpha-1 antitrypsin, | 0.91 | 0.92 (rounded | 9 |
| Example 9 | Kawasaki | C Reactive Protein, | | from 0.9212) | |
| | Disease | N-terminal prohormone | | | |
| | | of brain natriuretic | | | |
| | | peptide, thyroxine- | | | |
| | | binding globulin | | | |
| KDA 043 | Diagnosis for | C Reactive Protein, matrix | 0.95 | 0.96 (rounded | 10 |
| Example 10 | Kawasaki | metalloproteinase-9, | | from 0.9586) | |
| | Disease | N-terminal prohormone | | | |
| | | of brain natriuretic | | | |
| | | peptide, thyroxine- | | | |
| | | binding globulin | | | |
| KDA U007 | Diagnosis for | C Reactive Protein, | 0.91 | 0.92 (rounded | 11 |
| Example 11 | Kawasaki | N-terminal prohormone | | from 0.918) | |
| | Disease | of brain natriuretic | | | |
| | | peptide, T uptake | | | |

### Example 13: Relationship of BNP and NT-proBNP

B-type natriuretic peptide (BNP) and N-terminal B-type natriuretic peptide (NT-proBNP) have been shown to clinically assist in the diagnosis and management of heart failure (HF) and in determining patient prognosis. In response to myocardial wall stretch, pre-proBNP is synthesized and processed to proBNP; which is further processed to the biologically inactive NT-proBNP fragment and the biologically active BNP fragment. Since BNP and NT-proBNP can be measured and are elevated in patients with HF, both are useful adjuncts to clinical evaluation. In fact, the studies for BNP and NT-proBNP demonstrated similar sensitivity and specificity for ruling in and ruling out HF. Either protein can be used as part of the Kawasaki Disease panel and in the algorithm.

### REFERENCES

1. Singh et al. International Journal of Rheumatic Diseases 2018; 21: 36-44.
2. McCrindle, et al. Diagnosis, Treatment, and Long-Term Management of Kawasaki Disease A Scientific Statement for Health Professionals From the American Heart Association Circulation. 2017; 135:e927-e999. DOI: 10.1161/CIR.0000000000000484 Apr. 25, 2017.pp.e927-e999.

1. A method of determining Kawasaki disease in a subject, comprising:
(i) providing a biological sample from a subject suspected of having Kawasaki disease;
(ii) applying the biological sample to an analytical device to:
(a) detect a concentration of at least two protein markers in the biological sample;
(b) calculate the concentration of the at least two protein markers against a synthetic quantification standard to produce a protein marker concentration;
(c) log transform the concentration of the at least two protein markers to conform to a normal distribution; and
(d) normalize the log-transformed concentrations of the at least two protein markers to an established range and scale,
wherein the at least two protein markers are selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor;
(iii) calculating a diagnostic score using an algorithm that applies different weightings to the transformed, normalized concentration of protein markers determined in step (ii);
(iv) classifying the diagnostic score as a positive, intermediate, or negative score; and
(v) determining Kawasaki disease in the subject as indicated by the diagnostic score.

2. The method of claim 1, further comprising determining the status of at least one clinical variable for the subject, wherein the clinical variable is selected from age, race, fever ≥38.1° C., suspicion of Kawasaki disease, and persistent fever for 3 or more days.

3. The method of claim 2, further comprising calculating a diagnostic score using an algorithm that applies different weightings to the status of the clinical variable(s) determined in step (iii).

4. The method of claim 1, further comprising treating the subject based on the positive, intermediate, or negative score, wherein the treatment comprises a therapeutic intervention regimen.

5. The method of claim 1, wherein the biological sample is a blood sample.

6. The method of claim 2, wherein the at least two protein markers are selected from alpha-1 antitrypsin, C Reactive Protein, interleukin-1 beta, matrix metalloproteinase-9, N-terminal prohormone of brain natriuretic peptide, periostin, T3, T4, T3 uptake, T4 uptake, BNP, and thyroxine-binding globulin; and wherein the clinical variable is selected from age, race, fever equal to or greater than 38.1° C., and persistent fever for 3 or more days.

7. The method of claim 1 wherein the at least two protein markers are C Reactive Protein and N-terminal prohormone of brain natriuretic peptide.

8. The method of claim 1 wherein the at least two protein markers are C Reactive Protein, N-terminal prohormone of brain natriuretic peptide, and periostin.

9. The method of claim 1 wherein the at least two protein markers are alpha-1 antitrypsin, C Reactive Protein, and N-terminal prohormone of brain natriuretic peptide.

10. The method of claim 1 wherein the at least two protein markers are C Reactive Protein, matrix metalloproteinase-9, and N-terminal prohormone of brain natriuretic peptide.

11. The method of claim 1 wherein the at least two protein markers are alpha-1 antitrypsin, C Reactive Protein, matrix metalloproteinase-9, and N-terminal prohormone of brain natriuretic peptide.

12. The method of claim 1 wherein the at least two protein markers are C Reactive Protein, interleukin-1 beta, and N-terminal prohormone of brain natriuretic peptide.

13. The method of claim 1 wherein the at least two protein markers are alpha-1 antitrypsin, C Reactive Protein, interleukin-1 beta, N-terminal prohormone of brain natriuretic peptide.

14. The method of claim 1 wherein the at least two protein markers are C Reactive Protein, N-terminal prohormone of brain natriuretic peptide and thyroxine-binding globulin.

15. The method of claim 1 wherein the at least two protein markers are alpha-1 antitrypsin, C Reactive Protein, N-terminal prohormone of brain natriuretic peptide and thyroxine-binding globulin.

16. The method of claim 1 wherein the at least two protein markers are C Reactive Protein, matrix metalloproteinase-9, N-terminal prohormone of brain natriuretic peptide and thyroxine-binding globulin.

17. The method of claim 1 wherein the at least two protein markers are C Reactive Protein, N-terminal prohormone of brain natriuretic peptide and T uptake.

18. The method of claim 17, wherein the T uptake is T3 uptake or T4 uptake.

19. The method of claim 1 wherein a positive diagnostic score in the subject facilitates a determination by a medical practitioner of the need for one or more interventions selected from administration of pharmacological agents, echocardiography, and avoidance of high dose acetylsalicylic acid (ASA) in patients with concomitant active infection with varicella or influenza.

20. The method of claim 19, wherein the pharmacological agents are one or more of intravenous immunoglobulin, acetylsalicylic acid (ASA), methylprednisolone, and infliximab.

21. The method of claim 1 wherein an intermediate diagnostic score in the subject facilitates a determination by a medical practitioner of the need for one or more interventions selected from ongoing monitoring and further testing.

22. The method of claim 1 wherein a negative diagnostic score in the subject facilitates a determination by a medical practitioner of the need for one or more interventions selected from further testing and differential diagnosis of other diseases.

23. The method of claim 1, further comprising one or more protein markers that were previously unassociated with Kawasaki disease.

24. The method of claim 1, wherein the method further comprises specifically excluding one or more protein markers from claim 1.

25. A method of administering a therapeutic intervention to a subject suspected of having Kawasaki disease comprising:
(i) determining the subject's protein marker profile for a panel of protein markers comprising at least two protein markers selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor, optionally, determining the status of at least one clinical variable for the subject, wherein the clinical variable is age, race, fever ≥38.1° C., suspicion of Kawasaki disease, or persistent fever for 3 or more days;
(ii) assigning a score to the subject based on the protein marker profile in (i) and optionally the clinical value status in (ii) wherein the score is classified as a positive, intermediate, or negative score, said score algorithmically-derived from normalized and mathematically transformed concentrations of protein markers in the subject's sample and optionally, the status of at least one clinical variable; and
(iii) administering to the subject a therapeutic intervention based on the positive, intermediate or negative score.

26. A method of detecting two or more protein markers in a subject that is suspected of having Kawasaki disease, the method comprising:
(i) selecting a subject that is suspected of having Kawasaki disease;
(ii) providing a biological sample from the subject;
(iii) applying the biological sample to an analytical device;
(iv) detecting the concentration of at least two protein markers selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor; calculating a diagnostic score using an algorithm that applies different weightings to the concentration of protein markers determined in step (ii) and, optionally, the status of the clinical variable(s) determined in step (iii);
(vi) classifying the diagnostic score as a positive, intermediate, or negative score; and
(vii) determining Kawasaki disease in a subject as indicated by the diagnostic score.

27. The method of claim 26 wherein step (iv) comprises:
(a) calculating the concentration of the at least two protein markers against a synthetic quantification standard to produce a protein marker concentration;
(b) log transforming the concentration of the at least two protein markers to conform to a normal distribution; and
(c) normalizing the log-transformed concentrations of the at least two protein markers to an established range and scale.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The method of claim 1, wherein the subject has been diagnosed with SARS-CoV-2 infection.

46. The method of claim 1, wherein the subject has been diagnosed with Multi-System Inflammatory Syndrome in Children (MIS-C).

47. A panel for the diagnosis of Kawasaki disease, comprising target-binding agents that bind at least two protein markers selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor, a synthetic standard, and optionally, at least one clinical variable selected from age, race, fever ≥38.1° C., suspicion of Kawasaki disease, and persistent fever for 3 or more days.

48. (canceled)

49. A diagnostic kit comprising a panel according to claim 47.

50. (canceled)

51. (canceled)

52. A method for treating a patient with a fever or suspected of having Kawasaki Disease with intervention and additional testing, comprising:
(i) determining whether the patient suffers from Kawasaki Disease by: obtaining or having obtained a biological sample from the patient; performing or having performed a biomarker assay on the biological sample wherein the biomarker is selected from alpha-1 antitrypsin, C Reactive Protein, interleukin-1 beta, matrix metalloproteinase-9, N-terminal prohormone of brain natriuretic peptide, periostin thyroxine-binding globulin and T uptake; and calculating a diagnostic score based on a weighted level of each biomarker; and
(ii) if the patient has a positive diagnostic score, then performing additional testing or one or more interventions selected from administration of pharmacological agents, echocardiography, and avoidance of high dose acetylsalicylic acid (ASA) in patients with concomitant active infection with varicella or influenza, or enrolling in a clinical trial; if the patient has an intermediate score, then the need for one or more interventions selected from ongoing monitoring and further testing; and if the patient has a negative score, then the need for one or more of further testing and differential diagnosis of other diseases.

53. (canceled)

54. A method of determining Multi-System Inflammatory Syndrome in Children (MIS-C) in a subject, comprising:
(vi) providing a biological sample from a subject suspected of having MIS-C;
(vii) applying the biological sample to an analytical device to:
(a) detect a concentration of at least two protein markers in the biological sample;
(b) calculate the concentration of the at least two protein markers against a synthetic quantification standard to produce a protein marker concentration;
(c) log transform the concentration of the at least two protein markers to conform to a normal distribution; and
(d) normalize the log-transformed concentrations of the at least two protein markers to an established range and scale,
wherein the at least two protein markers are selected from Alpha-1 Antitrypsin, Alpha-1-Microglobulin, Ang-1, Apolipoprotein(a), Beta-2-Microglobulin, Brain-Derived Neurotrophic Factor, B-type natriuretic peptide (BNP), CD163, CD5 Antigen-like, Clusterin, Complement C3, C-Reactive Protein, Cystatin-C, Eotaxin-1, Factor VII, Fibrinogen, Growth/differentiation factor 15, Haptoglobin, Immunoglobulin A, Immunoglobulin M, Intercellular Adhesion Molecule 1, Interleukin-1 alpha, Interleukin-1 beta, Interleukin-1 receptor antagonist, Interleukin-12 Subunit p40, Interleukin-12 Subunit p′70, Interleukin-17, Kidney Injury Molecule-1 (KIM-1), Matrix Metalloproteinase-3, Matrix Metalloproteinase-9, Midkine, N-terminal B-type natriuretic peptide (NT-proBNP), Osteocalcin, Osteopontin, Periostin, P-Selectin, Serum Amyloid P-Component, ST2, Stem Cell Factor, T3 Uptake, T4 Uptake, Thyroid-stimulating hormone (TSH), thyroxine (T4), Thyroxine-Binding Globulin (TBG), Triiodothyronine (T3), Vascular Endothelial Growth Factor, Vitamin D-Binding Protein, and von Willebrand Factor;
(viii) calculating a diagnostic score using an algorithm that applies different weightings to the transformed, normalized concentration of protein markers determined in step (ii);
(ix) classifying the diagnostic score as a positive, intermediate, or negative score; and
(x) determining MIS-C in the subject as indicated by the diagnostic score.

55. The method of claim 1, further comprising determining the status of at least one clinical variable for the subject, wherein the clinical variable is selected from age, race, fever ≥38.1° C., suspicion of SARS-CoV-2 or MIS-C and/or diagnosis of infection with SARS-CoV-2, and persistent fever for 3 or more days.

56. The method of claim 2, further comprising calculating a diagnostic score using an algorithm that applies different weightings to the status of the clinical variable(s) determined in step (iii).

57. The method of claim 1, further comprising treating the subject based on the positive, intermediate, or negative score, wherein the treatment comprises a therapeutic intervention regimen.
